(19) United States
(12) Patent Application Publication
(10) Pub. No.: US 2026/0019714 A1
(43) Pub. Date: Jan. 15, 2026

(54) CONTROL DEVICE, ENDOSCOPE SYSTEM, OPERATION METHOD OF CONTROL DEVICE, AND PROGRAM

(71) Applicant: FUJIFILM Corporation, Tokyo (JP)

(72) Inventor: Teppei YAMAKI, Kanagawa (JP)

(73) Assignee: FUJIFILM Corporation, Tokyo (JP)

(21) Appl. No.: 19/258,953

(22) Filed: Jul. 3, 2025

(30) Foreign Application Priority Data
Jul. 9, 2024 (JP) 2024-110545

(51) Int. Cl. H04N 23/74; A61B 1/00; A61B 1/06; H04N 23/50; H04N 23/71; H04N 23/72; H04N 23/73

(52) U.S. Cl. CPC H04N 23/74; A61B 1/0019; A61B 1/0655; H04N 23/555; H04N 23/71; H04N 23/72; H04N 23/73

(57) ABSTRACT

A processor performs brightness control of bringing brightness of an image obtained by imaging an inside of a body with an endoscope in a state in which the inside of the body is irradiated with light, closer to target brightness. The brightness control is classified into first brightness control and second brightness control. The first brightness control is performed in a case in which an instrument is not mounted on a distal end portion of the endoscope. The second brightness control is performed in a case in which the instrument is mounted on the distal end portion of the endoscope, and is different from the first brightness control.

### CROSS-REFERENCE TO RELATED APPLICATION

This application claims priority under 35 USC 119 from Japanese Patent Application No. 2024-110545, filed on Jul. 9, 2024, the disclosure of which is incorporated by reference herein.

### BACKGROUND

### 1. Technical Field

The present disclosure relates to a control device, an endoscope system, an operation method of a control device, and a program.

### 2. Related Art

JP2009-060237A discloses an imaging apparatus comprising an imaging unit that images a subject to obtain a captured image, a comparison unit, a light measurement unit, and a condition adjustment unit. In the imaging apparatus disclosed in JP2009-060237A, the comparison unit compares brightness in a first portion including a center of the captured image obtained by the imaging unit with brightness in a second portion closer to a side edge in the captured image with respect to the first portion. The light measurement unit measures an overall brightness of the captured image by mixing average brightness of the captured image obtained by the imaging unit and brightness in a high-brightness portion in the captured image with a weight that is heavier for the brightness of the high-brightness portion as the brightness of the first portion is brighter than the brightness of the second portion in the comparison of the comparison unit. The condition adjustment unit adjusts an imaging condition that affects brightness of the captured image based on the brightness measured by the light measurement unit.

### SUMMARY

One embodiment according to the present disclosure provides a control device, an endoscope system, an operation method of a control device, and a program that can prevent an image from becoming dark overall by brightness control that is performed due to the image becoming bright due to an influence of halation that occurs in a case in which an instrument is mounted on a distal end portion of an endoscope.

A first aspect according to the present disclosure relates to a control device used for an endoscope having a distal end portion for emitting light, the control device comprising: a processor, in which the processor performs brightness control of bringing brightness of an image obtained by imaging an inside of a body with the endoscope in a state in which the inside of the body is irradiated with the light, closer to target brightness, the brightness control is classified into first brightness control and second brightness control, the first brightness control is performed in a case in which an instrument is not mounted on the distal end portion, and the second brightness control is performed in a case in which the instrument is mounted on the distal end portion, and is different from the first brightness control.

A second aspect according to the present disclosure relates to the control device according to the first aspect, in which the first brightness control or the second brightness control is performed based on a result of comparing a light measurement value of a first image obtained by imaging the inside of the body with the endoscope in a state in which the inside of the body is irradiated with the light, with a target light measurement value, and a degree of increase in brightness of a second image obtained later than the first image by imaging the inside of the body with the endoscope in a state in which the inside of the body is irradiated with the light is greater in the second brightness control than in the first brightness control.

A third aspect according to the present disclosure relates to the control device according to the second aspect, in which the brightness of the second image is increased by lengthening an exposure time with the endoscope in a case in which the imaging for obtaining the second image is performed, increasing a light quantity of the light in a case in which the imaging for obtaining the second image is performed, or increasing a degree of an influence of image processing for brightness adjustment on the second image in a case in which the image processing is performed on the second image.

A fourth aspect according to the present disclosure relates to the control device according to the third aspect, in which the image processing is performed on a specific region of the second image, and the specific region is determined in accordance with the instrument.

A fifth aspect according to the present disclosure relates to the control device according to any one of the second to fourth aspects, in which the brightness control is performed based on a plurality of light measurement values obtained by measuring light of the first image using a plurality of light measurement methods, and a degree of use of the plurality of light measurement values is different between the first brightness control and the second brightness control.

A sixth aspect according to the present disclosure relates to the control device according to the fifth aspect, in which the plurality of light measurement values include an average light measurement value of the first image, and a degree of use of the average light measurement value in the second brightness control is greater than a degree of use of the average light measurement value in the first brightness control.

A seventh aspect according to the present disclosure relates to the control device according to any one of the second to sixth aspects, in which the brightness control is performed based on the light measurement value of the first image, the light measurement value is obtained for each divided region in which the first image is divided by a division method different between the first brightness control and the second brightness control, and magnitude of the light measurement values used in the first brightness control and the second brightness control is magnitude in accordance with the divided region.

An eighth aspect according to the present disclosure relates to the control device according to the seventh aspect, in which the division method used in the second brightness control is determined in accordance with the instrument.

A ninth aspect according to the present disclosure relates to the control device according to any one of the second to eighth aspects, in which the brightness control is performed based on the light measurement value of the first image, and the light measurement value used in the second brightness control is obtained from an inner region of the first image as compared with the light measurement value used in the first brightness control.

A tenth aspect according to the present disclosure relates to the control device according to any one of the first to ninth aspects, in which the endoscope is a variable magnification endoscope, and the second brightness control is valid in a case in which a magnification of the variable magnification endoscope is a first magnification, and is invalid in a case in which the magnification is a second magnification greater than the first magnification or a third magnification less than the first magnification.

An eleventh aspect according to the present disclosure relates to the control device according to any one of the first to tenth aspects, in which a valid state in which the second brightness control is valid and an invalid state in which the second brightness control is invalid are switched in accordance with a type of the endoscope.

A twelfth aspect according to the present disclosure relates to the control device according to any one of the first to eleventh aspects, in which the brightness of the image is changed stepwise in a case in which the first brightness control and the second brightness control are switched from one to the other.

A thirteenth aspect according to the present disclosure relates to the control device according to the twelfth aspect, in which the stepwise change in the brightness of the image is achieved by lengthening, stepwise, an exposure time with the endoscope in a case in which the imaging for obtaining the image is performed, increasing, stepwise, a light quantity of the light in a case in which the imaging for obtaining the image is performed, or increasing, stepwise, a degree of an influence of image processing for brightness adjustment on the image in a case in which the image processing is performed on the image.

A fourteenth aspect according to the present disclosure relates to the control device according to any one of the first to thirteenth aspects, in which a content of the second brightness control varies in accordance with a type of the instrument.

A fifteenth aspect according to the present disclosure relates to the control device according to any one of the first to fourteenth aspects, in which the instrument is a tubular hood having an opening.

A sixteenth aspect according to the present disclosure relates to the control device according to any one of the first to fifteenth aspects, in which the instrument has optical characteristics that cause halation caused by the light.

A seventeenth aspect according to the present disclosure relates to the control device according to any one of the first to sixteenth aspects, in which the target brightness used in the second brightness control is higher than the target brightness used in the first brightness control.

An eighteenth aspect according to the present disclosure relates to an endoscope system comprising: the control device according to any one of the first to seventeenth aspects; and the endoscope.

A nineteenth aspect according to the present disclosure relates to an operation method of a control device used for an endoscope having a distal end portion for emitting light, the operation method comprising: performing brightness control of bringing brightness of an image obtained by imaging an inside of a body with the endoscope in a state in which the inside of the body is irradiated with the light, closer to target brightness, in which the brightness control is classified into first brightness control and second brightness control, the first brightness control is performed in a case in which an instrument is not mounted on the distal end portion, and the second brightness control is performed in a case in which the instrument is mounted on the distal end portion, and is different from the first brightness control.

A twentieth aspect according to the present disclosure relates to a program causing a computer used for an endoscope having a distal end portion for emitting light, to execute a process comprising: performing brightness control of bringing brightness of an image obtained by imaging an inside of a body with the endoscope in a state in which the inside of the body is irradiated with the light, closer to target brightness, in which the brightness control is classified into first brightness control and second brightness control, the first brightness control is performed in a case in which an instrument is not mounted on the distal end portion, and the second brightness control is performed in a case in which the instrument is mounted on the distal end portion, and is different from the first brightness control.

### BRIEF DESCRIPTION OF THE DRAWINGS

Exemplary embodiments of the technology of the disclosure will be described in detail based on the following figures, wherein:

FIG. 1 is a conceptual diagram showing an aspect example in which an endoscope system is used by a doctor;

FIG. 2 is a block diagram showing an example of a hardware configuration of an electrical system of the endoscope system;

FIG. 3 is a conceptual diagram showing an example of a display aspect of a moving image in a case in which a hood is not mounted and an example of a display aspect of a moving image in a case in which the hood is mounted;

FIG. 4 is a block diagram showing an example of main functions of a processor;

FIG. 5 is a conceptual diagram showing an example of processing contents of a determination unit;

FIG. 6 is a conceptual diagram showing an example of processing contents of a controller;

FIG. 7A is a flowchart showing an example of a flow of brightness control processing.

FIG. 7B is a continuation of the flowchart shown in FIG. 7A;

FIG. 8 is a conceptual diagram showing a first modification example of the processing contents of the controller;

FIG. 9 is a conceptual diagram showing a second modification example of the processing contents of the controller;

FIG. 10 is a conceptual diagram showing an aspect example in which brightness of a frame is changed stepwise from first brightness to second brightness;

FIG. 11 is a conceptual diagram showing an aspect example in which the brightness of the frame is changed monotonically from the first brightness to the second brightness;

FIG. 12 is a conceptual diagram showing a first modification example of the processing contents of the determination unit;

FIG. 13 is a conceptual diagram showing a second modification example of the processing contents of the determination unit;

FIG. 14 is a conceptual diagram showing a third modification example of the processing contents of the determination unit;

FIG. 15 is a conceptual diagram showing a fourth modification example of the processing contents of the determination unit;

FIG. 16 is a conceptual diagram showing a fifth modification example of the processing contents of the determination unit;

FIG. 17 is a conceptual diagram showing a third modification example of the processing contents of the controller;

FIG. 18 is a conceptual diagram showing a fourth modification example of the processing contents of the controller; and

FIG. 19 is a conceptual diagram showing a sixth modification example of the processing contents of the determination unit.

### DETAILED DESCRIPTION

Hereinafter, an example of embodiments of a control device, an endoscope system, an operation method of a control device, and a program according to the present disclosure will be described with reference to the accompanying drawings. It should be noted that the present disclosure can also be applied to a program and a computer program product.

First, the terms used in the following description will be described.

CPU is an abbreviation for “central processing unit”. GPU is an abbreviation for “graphics processing unit”. GPGPU is an abbreviation for “general-purpose computing on graphics processing units”. APU is an abbreviation for “accelerated processing unit”. TPU is an abbreviation for “tensor processing unit”. RAM is an abbreviation for “random-access memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “universal serial bus”. EL is an abbreviation for “electro-luminescence”. CMOS is an abbreviation for “complementary metal oxide semiconductor”. CCD is an abbreviation for “charge coupled device”. I/F is an abbreviation for “interface”. 5G is an abbreviation for “5th generation mobile communication system”. IC is an abbreviation for “integrated circuit”.

Hereinafter, a processor with a reference numeral (hereinafter, simply referred to as “processor”) may be one physical or virtual computing device or a combination of a plurality of physical or virtual computing devices. The processor may be one type of computing device or may be a combination of a plurality of types of computing devices. Examples of the computing device include a CPU, a GPU, a GPGPU, an APU, and a TPU.

Hereinafter, a memory with a reference numeral is a memory, such as a RAM, that temporarily stores information, and is used by the processor as a work memory.

Hereinafter, a storage with a reference numeral is one or a plurality of non-volatile storage devices that store various programs, various parameters, and the like. Examples of the non-volatile storage device include a flash memory, a magnetic disk, and a magnetic tape. Another example of the storage is a cloud storage.

In the following embodiment, an external I/F with a reference numeral controls the transmission and reception of various types of information among a plurality of devices connected to each other. Examples of the external I/F include a USB interface. A communication I/F including a communication processor, an antenna, and the like may be applied to the external I/F. The communication I/F controls communication among a plurality of computers. Examples of a communication standard applied to the communication I/F include a wireless communication standard including 5G, Wi-Fi (registered trademark), and Bluetooth (registered trademark).

Hereinafter, “A and/or B” is synonymous with “at least one of A or B”. That is, “A and/or B” may mean only A, only B, or a combination of A and B. In addition, in the present specification, the same concept as “A and/or B” is applied to a case in which the connection of three or more matters is expressed by “and/or”.

Hereinafter, the “same shape” refers to the same shape in the sense of including an error that is generally allowed in the technical field to which the present disclosure belongs and that does not contradict the gist of the present disclosure, in addition to the completely same shape.

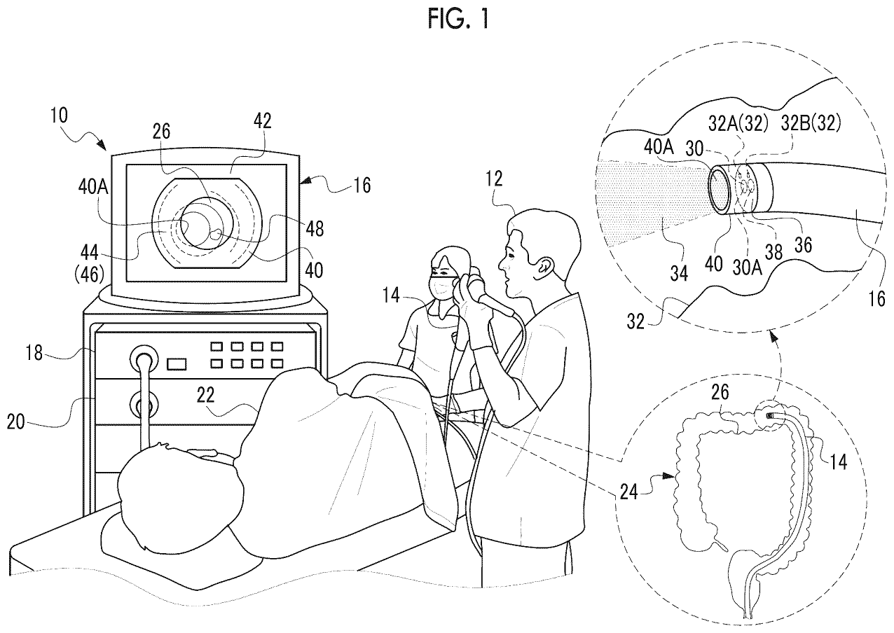

FIG. 1 shows an example of an aspect in which an endoscope system 10 is used. As shown in FIG. 1, an endoscope system 10 is used by a doctor 12 in an endoscopy and the like. The endoscope system 10 comprises an endoscope 14, a display device 16, a control device 18, and a light source device 20.

The endoscope system 10 is a modality for a doctor 12 to perform medical care on a large intestine 24 included inside a body of a subject 22 (for example, a patient) using the endoscope 14. Here, although a lower endoscopy is shown as an example for the purpose of medical care for the large intestine 24, this is merely an example, and the present disclosure can also be applied to an endoscopy performed for the purpose of medical care for a luminal organ other than the large intestine 24, such as an upper endoscopy.

The endoscope 14 is used by the doctor 12, and is inserted into the large intestine 24 of the subject 22. The endoscope system 10 causes the endoscope 14 inserted into the large intestine 24 to image the inside of the large intestine 24 (for example, a region including an intestinal wall 26), and performs various medical treatments on the large intestine 24 as necessary.

The endoscope 14 comprises a camera 30 that images the inside of the large intestine 24, and an illumination device 32 that irradiates the inside of the large intestine 24 with light 34 through irradiation ports 32A and 32B. The camera 30 includes an objective lens 30A. The objective lens 30A is provided at a distal end portion 36 (a distal end surface of the endoscope 14 in the example shown in FIG. 1) of the endoscope 14. In addition, the irradiation ports 32A and 32B are provided at the distal end portion 36 (the distal end surface of the endoscope 14 in the example shown in FIG. 1). Subject light, which is reflected light obtained by being reflected in a region including the intestinal wall 26 by the light 34 emitted from the irradiation ports 32A and 32B in the large intestine 24, is incident on the objective lens 30A. The camera 30 images the subject light incident on the objective lens 30A.

A treatment tool opening 38 is provided at the distal end portion 36 of the endoscope 14 (the distal end surface of the endoscope 14 in the example shown in FIG. 1). The treatment tool opening 38 is an opening for allowing a treatment tool (not shown) to be taken out from the distal end portion 36 (the distal end surface of the endoscope 14 in the example shown in FIG. 1). Examples of the treatment tool that is delivered from the treatment tool opening 38 include a gripping forceps, a biopsy needle, a papillotomy knife, a snare, a catheter, a guide wire, a cannula, and/or a biopsy needle with a guide sheath. Further, the treatment tool opening 38 is also used as a suction port for suctioning blood, internal contaminants, and the like and a sending-out port for sending out fluid.

A hood 40 (in the present embodiment, as an example, a transparent hood) having translucency is attachably and detachably mounted on the distal end portion 36. The hood 40 is an example of an “instrument” and a “hood” according to the present disclosure. The hood 40 is an instrument having optical characteristics that cause halation caused by the light 34. Here, although the hood 40 is shown as an example, in addition to the hood 40, an instrument (for example, an instrument having optical characteristics that cause halation caused by the light 34) such as an external treatment tool is attachably and detachably mounted on the distal end portion 36.

The shape of the hood 40 is a tubular shape (here, as an example, a cylindrical shape). The hood 40 is used for maintaining an appropriate distance between the objective lens 30A, the irradiation ports 32A and 32B, and the treatment tool opening 38 and the subject (for example, an observation target in the intestinal wall 26) and to secure a good visual field. The hood 40 may also be used to support an operation of the treatment tool. The hood 40 has an opening 40A having a circular shape in front view. By mounting the hood 40 on the distal end portion 36, the objective lens 30A, the irradiation ports 32A and 32B, and the treatment tool opening 38 face the opening 40A. Therefore, the opening 40A is included in an angle of view of the camera 30, the region including the intestinal wall 26 is irradiated with the light 34 emitted from the irradiation ports 32A and 32B through the opening 40A, and the treatment tool is taken in and out of the opening 40A through the treatment tool opening 38. In addition, in a case in which the hood 40 is mounted on the distal end portion 36, the region including the intestinal wall 26 is irradiated with the light 34 emitted from the irradiation ports 32A and 32B via the hood 40. In the example shown in FIG. 1, a form example is shown in which the light 34 is emitted from the opening 40A, but the hood 40 has translucency, and thus the light 34 emitted from the irradiation ports 32A and 32B is transmitted through the wall surface of the hood 40. Further, the subject light is also transmitted through the wall surface of the hood 40 and is incident on the objective lens 30A.

The control device 18 controls the entire endoscope system 10. The control device 18 is used, for example, for the endoscope 14, the display device 16, the light source device 20, and the like, and the endoscope 14, the display device 16, the light source device 20, and the like are controlled by the control device 18.

The display device 16 displays various types of information on a screen 42 under the control of the control device 18. Examples of the display device 16 include a liquid crystal display and an EL display. A tablet terminal with a display may be used instead of the display device 16 or together with the display device 16.

The light source device 20 generates the light 34 and supplies the generated light 34 to the illumination device 32, under the control of the control device 18. A light guide (not shown) is built in the illumination device 32, and the light 34 supplied from the light source device 20 is emitted from the irradiation ports 32A and 32B via the light guide.

The camera 30 generates a moving image 44 by imaging the observation target in the intestinal wall 26 under the control of the control device 18. The moving image 44 includes a plurality of frames 46 in time series. The control device 18 acquires the moving image 44 generated by the camera 30, and performs various types of image processing on the acquired moving image 44. Then, the control device 18 outputs various types of information including the moving image 44 on which the image processing has been performed, to the display device 16. As a result, various types of information including the moving image 44 are displayed on the screen 42 of the display device 16. The plurality of frames 46 in time series are displayed on the screen 42 at a predetermined frame rate. Examples of the predetermined frame rate include 15 frames/second, 30 frames/second, and 60 frames/second.

In the example shown in FIG. 1, the moving image 44 obtained by performing the imaging with the camera 30 in a state in which the hood 40 is mounted on the distal end portion 36 is displayed on the screen 42. The hood 40 including the opening 40A enters the angle of view of the camera 30. Therefore, in addition to the intestinal wall 26 (the intestinal wall 26 including a lesion 48 in the example shown in FIG. 1), the moving image 44 also shows the hood 40 along with the opening 40A. Further, the intestinal wall 26 is shown through the opening 40A in the moving image 44 displayed on the screen 42. In addition, the intestinal wall 26 is also shown in the moving image 44 displayed on the screen 42, through the wall surface of the hood 40. Therefore, in a case in which the hood 40 is mounted on the distal end portion 36, the doctor 12 can observe the intestinal wall 26 from the inside and the outside of the opening 40A through the screen 42.

It should be noted that the endoscope system 10 in the present embodiment is an example of an “endoscope system” according to the present disclosure. In addition, the endoscope 14 in the present embodiment is an example of an “endoscope” according to the present disclosure. In addition, the control device 18 in the present embodiment is an example of a “control device” according to the present disclosure. In addition, the light 34 in the present embodiment is an example of “light” according to the present disclosure. In addition, the distal end portion 36 in the present embodiment is an example of a “distal end portion” according to the present disclosure. In addition, the camera 30 in the present embodiment is an example of a “camera” according to the present disclosure. In addition, the hood 40 in the present embodiment is an example of a “hood” according to the present disclosure. In addition, the opening 40A in the present embodiment is an example of an “opening” according to the present disclosure. In addition, the frame 46 in the present embodiment is an example of an “image” according to the present disclosure.

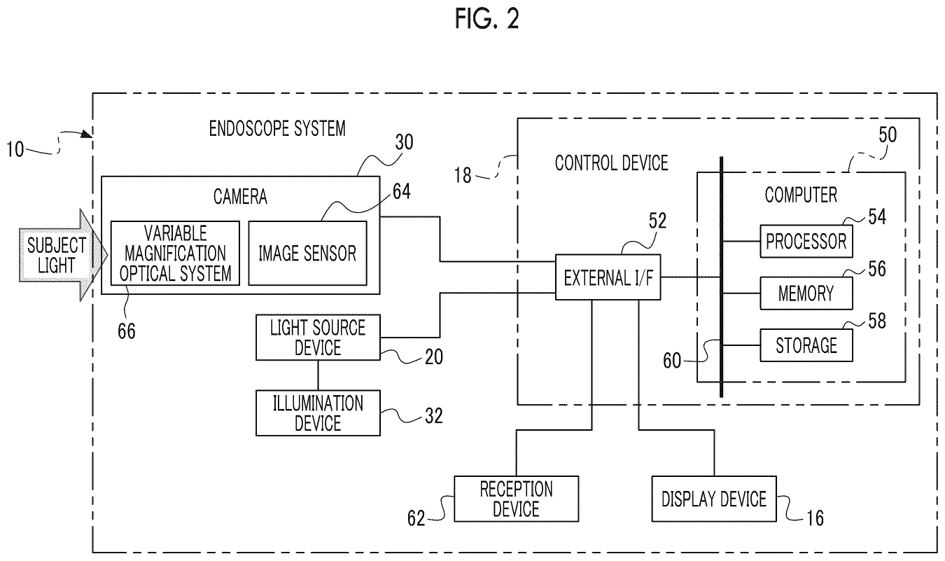

FIG. 2 shows an example of a hardware configuration of an electrical system of the endoscope system 10. As shown in FIG. 2, the control device 18 comprises a computer 50 and an external I/F 52. The computer 50 comprises a processor 54, a memory 56, and a storage 58. The processor 54, the memory 56, the storage 58, and the external I/F 52 are connected to a bus 60. The processor 54 controls the entire control device 18. The memory 56 and the storage 58 are used by the processor 54.

The external I/F 52 transmits and receives various types of information between one or more devices (hereinafter, also referred to as “external devices”) existing outside the control device 18 and the processor 54.

A reception device 62 is connected to the external I/F 52, as one of the external devices. Examples of the reception device 62 include a foot switch, a microphone, a touch panel, a keyboard, and/or a mouse. The processor 54 acquires an instruction received by the reception device 62 via the external I/F 52 and executes processing in accordance with the acquired instruction.

The display device 16 is connected to the external I/F 52, as one of the external devices. The processor 54 controls the display device 16 via the external I/F 52 such that various types of information (for example, the moving image 44 on which various types of image processing have been performed) are displayed on the display device 16.

The light source device 20 is connected to the external I/F 52, as one of the external devices, and the external I/F 52 controls the exchange of various types of information between the light source device 20 and the processor 54. The light source device 20 generates the light 34 (see FIG. 1) to supply the light 34 to the illumination device 32 under the control of the processor 54. The illumination device 32 emits the light 34 (see FIG. 1) supplied from the light source device 20.

The camera 30 is connected to the external I/F 52, as one of the external devices. The external I/F 52 controls the transmission and the reception of various types of information between the camera 30 and the processor 54. The camera 30 includes an image sensor 64 and a variable magnification optical system 66. That is, the endoscope 14 can be referred to as a variable magnification endoscope.

Examples of the image sensor 64 include a CMOS image sensor and a CCD image sensor. The variable magnification optical system 66 is an optical system that implements a so-called optical zoom, and operates under the control of the processor 54. The variable magnification optical system 66 is operated to change the magnification of the moving image 44 (see FIG. 1). That is, the frame 46 (see FIG. 1) is optically zoomed in or zoomed out. The image sensor 64 receives the subject light incident on the variable magnification optical system 66 and photoelectrically converts the received subject light to generate an electric signal in accordance with the subject light. A signal processing circuit (not shown) connected to the image sensor 64 is included in the camera 30. The signal processing circuit acquires the electric signal from the image sensor 64 and executes various types of signal processing including A/D conversion on the acquired electric signal to generate the frame 46 (see FIG. 1) in accordance with a predetermined frame rate (for example, a frame rate determined in advance, such as 15 frames/second, 30 frames/second, or 60 frames/second).

Each time the frame 46 (see FIG. 1) is generated by the camera 30 in accordance with the predetermined frame rate, the generated frame 46 is acquired by the processor 54. That is, the processor 54 acquires the moving image 44 (see FIG. 1) including the plurality of frames 46 in time series from the camera 30.

Here, although the form example has been described in which the frame 46 is generated by executing various types of signal processing on the electric signal in accordance with the subject light by the signal processing circuit of the camera 30, this is merely an example. For example, the control device 18 may include a signal processing circuit, and the signal processing circuit of the control device 18 may acquire the electric signal in accordance with the subject light from the image sensor 64 and execute various types of signal processing to generate the frame 46.

It should be noted that the computer 50 in the present embodiment is an example of a “computer” according to the present disclosure. In addition, the processor 54 in the present embodiment is an example of a “processor” according to the present disclosure.

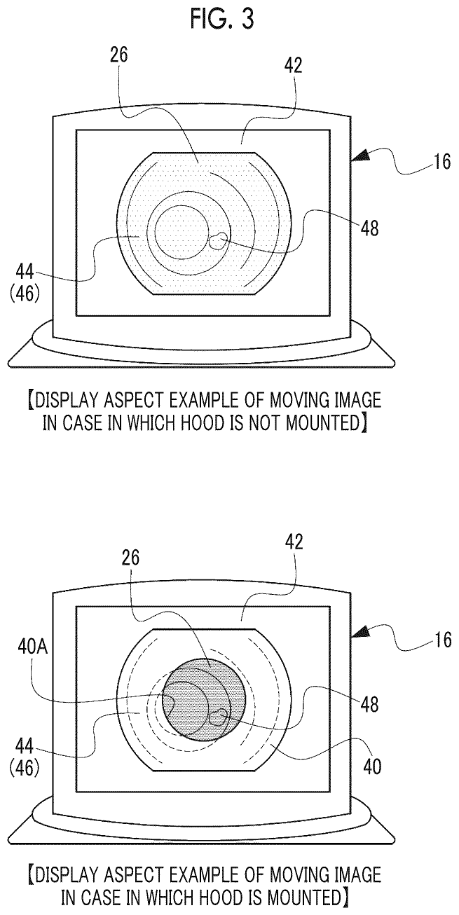

Meanwhile, each of the plurality of frames 46, which are included in the moving image 44 acquired from the camera 30 by the processor 54, is displayed on the screen 42 (see FIGS. 1 and 3) of the display device 16. For example, in a case in which the hood 40 is mounted on the distal end portion 36, as shown in FIG. 3 as an example, the hood 40 is shown in the screen 42.

In an endoscope system in the related art, in a case in which the hood 40 is shown in the frame 46, brightness control is performed to control the brightness in the frame 46 by performing control of an exposure time, control of a light quantity, and/or image processing on the frame 46 such that the brightness of the hood 40 included in the frame 46 is appropriate, rather than the observation target of the intestinal wall 26. Then, for example, in a case in which the hood 40 is mounted on the distal end portion of the endoscope, the frame 46 becomes dark overall due to brightness control that is performed due to the frame 46 becoming bright due to the influence of halation (that is, halation caused by the light 34) that occurs in the hood 40.

For example, in this case, the observation target (for example, an inner region of the opening 40A) located at a distant view from the hood 40 does not have appropriate brightness, which hinders the observation by the doctor 12. In addition, it goes without saying that the brightness of the frame 46 is different between a case in which the hood 40 is not mounted on the distal end portion 36 and a case in which the hood 40 is mounted on the distal end portion 36. For example, the frame 46 obtained in a case in which the hood 40 is mounted on the distal end portion 36 is darker overall than the frame 46 obtained in a case in which the hood 40 is not mounted on the distal end portion 36. This is because the hood 40 blocks a part of the subject light even in a case in which the hood 40 is a hood having translucency.

The doctor 12 gives an instruction to the endoscope system 10 to adjust the intensity of the light 34 or to switch the operation mode of the brightness control such that the brightness of the subject is appropriate depending on the hood 40 is not mounted on the distal end portion or the hood 40 is mounted on the distal end portion. However, it is troublesome for the doctor 12 to give an instruction to the endoscope system 10 for appropriately adjusting the brightness of the subject.

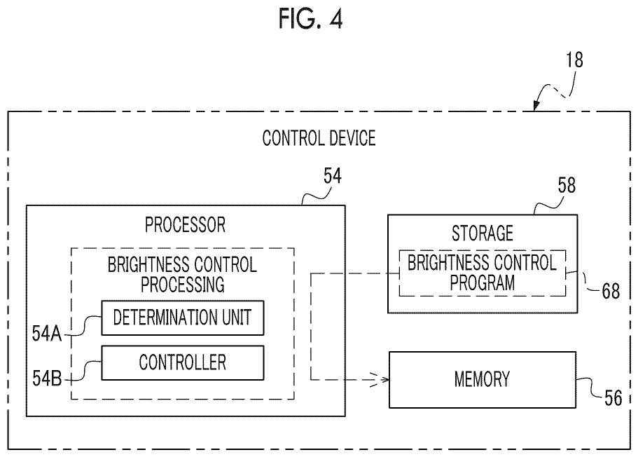

Therefore, in view of such circumstances, in the present embodiment, as shown in FIG. 4 as an example, the brightness control processing is executed by the processor 54 of the control device 18. It should be noted that FIG. 4 shows an example of an operation mode of the control device 18 for implementing the brightness control processing.

The storage 58 stores a brightness control program 68. In the present embodiment, the brightness control program 68 is an example of a “program” according to the present disclosure. The processor 54 executes the brightness control processing by reading out the brightness control program 68 from the storage 58 and executing the readout brightness control program 68 on the memory 56. The brightness control processing is implemented by the processor 54 operating as a determination unit 54A and a controller 54B in accordance with the brightness control program 68 executed on the memory 56.

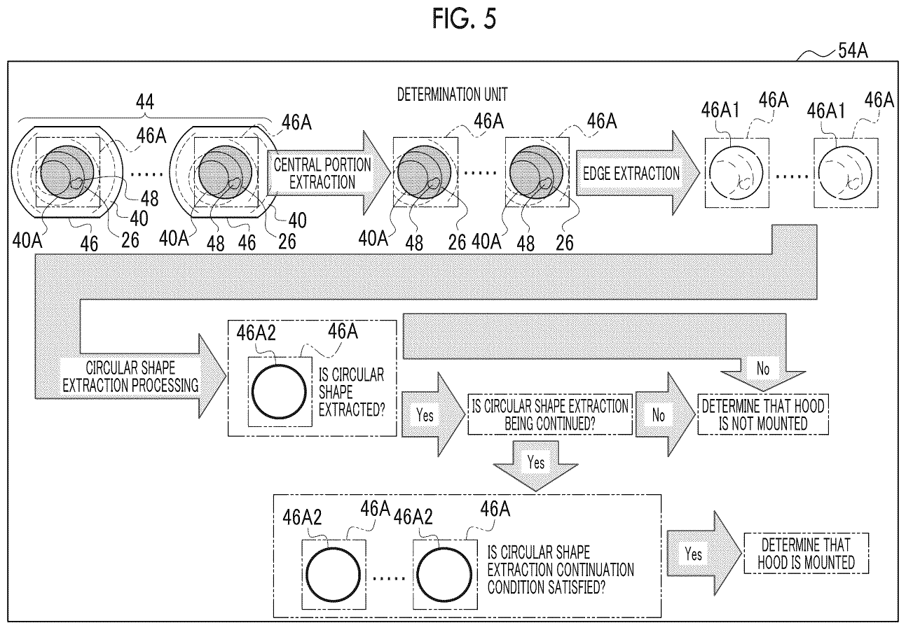

In the brightness control processing, whether or not the hood 40 is mounted on the distal end portion 36 is specified based on a feature value of the frame 46 and a condition (for example, a time condition and/or a distribution condition of brightness included in the frame 46, which is an example of the feature value of the frame 46) given to the feature value of the frame 46. Whether or not the hood 40 is mounted on the distal end portion 36 is specified by the determination unit 54A as shown in FIG. 5. Hereinafter, an example of the processing contents of the determination unit 54A will be described with reference to FIG. 5.

FIG. 5 shows an example of the processing contents of the determination unit 54A. As shown in FIG. 5, the determination unit 54A determines whether or not the hood 40 is mounted on the distal end portion 36 based on the frame 46 obtained by imaging the inside of the large intestine 24 with the camera 30 in a state in which the inside of the large intestine 24 is irradiated with the light 34. In order to realize this, first, the determination unit 54A extracts a central portion 46A from each of the plurality of frames 46 in time series included in the moving image 44. The reason for extracting the central portion 46A is that the central portion 46A is a portion in which a degree of attention by the doctor 12 is the highest, or the feature (for example, the opening 40A) of the hood 40 is likely to appear in the central portion 46A as compared with a portion of the frame 46 other than the central portion 46A. In the example shown in FIG. 5, a rectangular region of which a center coincides with the center of the frame 46 is shown as an example of the central portion 46A. The central portion 46A may be a region having a geometric shape other than a rectangular shape, such as a circular region, instead of a rectangular region.

The determination unit 54A extracts an edge 46A1, which is shown in the central portion 46A, from the central portion 46A. For example, the edge 46A1 is an image region of a high-frequency component determined in advance as a high-frequency component representing the edge 46A1 by a test using a real machine and/or a computer simulation.

The determination unit 54A executes circular shape extraction processing. The circular shape extraction processing is processing of extracting a circular shape 46A2 from the edge 46A1 by performing the Hough transformation on the edge 46A1 extracted from the central portion 46A. The reason for extracting the circular shape 46A2 is that the opening 40A has a circular shape. That is, in a case in which the shape (that is, the circular shape) of the opening 40A is included in the central portion 46A, it can be said that the hood 40 is mounted on the distal end portion 36.

The determination unit 54A determines whether or not the circular shape 46A2 is extracted from the edge 46A1. Here, in a case in which the determination unit 54A determines that the circular shape 46A2 is not extracted from the edge 46A1, the determination unit 54A determines that the hood 40 is not mounted on the distal end portion 36.

On the other hand, in a case in which the determination unit 54A determines that the circular shape 46A2 is extracted from the edge 46A1, the determination unit 54A determines whether or not the extraction of the circular shape 46A2 is being continued over the plurality of frames 46. Here, in a case in which the determination unit 54A determines that the extraction of the circular shape 46A2 is not being continued over the plurality of frames 46 (in other words, in a case in which the extraction of the circular shape 46A2 is interrupted), the determination unit 54A determines that the hood 40 is not mounted on the distal end portion 36. In addition, in a case in which the determination unit 54A determines that the extraction of the circular shape 46A2 is being continued over the plurality of frames 46, the determination unit 54A subsequently determines whether or not a circular shape extraction continuation condition is satisfied.

The circular shape extraction continuation condition refers to a condition in which the circular shape 46A2 is continuously extracted for a predetermined time (for example, a time designated in advance in a range of several seconds to several tens of seconds) or longer. A predetermined number of frames is included in the concept of the predetermined time. Examples of the predetermined number of frames include the number of frames (for example, 90 frames) designated in advance within a range of several tens of frames to several hundreds of frames. In a case in which the circular shape extraction continuation condition is satisfied, the determination unit 54A determines that the hood 40 is mounted on the distal end portion 36. In a case in which the circular shape extraction continuation condition is not satisfied, the determination unit 54A executes the above-described processing (that is, the series of processing from the processing of extracting the central portion 46A from the frame 46) on the next frame 46 again.

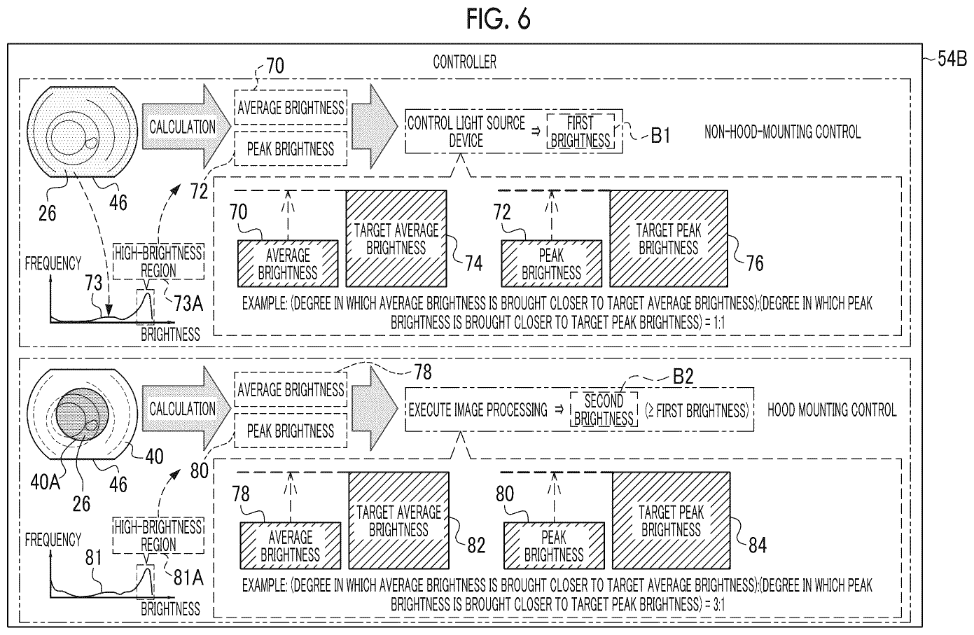

In the brightness control processing, the brightness control performed on the frame 46 is different between a case in which the determination unit 54A determines that the hood 40 is not mounted on the distal end portion 36 and a case in which the determination unit 54A determines that the hood 40 is mounted on the distal end portion 36. However, these types of control are common in that the control is performed for bringing the brightness of the frame 46 closer to target brightness. As shown in FIG. 6, the brightness control performed on the frame 46 is implemented by the controller 54B. Hereinafter, an example of the processing contents of the controller 54B will be described with reference to FIG. 6.

FIG. 6 shows an example of the processing contents of the controller 54B. As shown in FIG. 6, the controller 54B performs the brightness control that is the control of bringing the brightness of the frame 46 closer to the target brightness. The brightness control is classified into non-hood-mounting control and hood mounting control. The controller 54B selectively performs the non-hood-mounting control and the hood mounting control. The non-hood-mounting control is brightness control performed on the frame 46 in a case in which the determination unit 54A determines that the hood 40 is not mounted on the distal end portion 36. The hood mounting control is a brightness control performed on the frame 46 in a case in which the determination unit 54A determines that the hood 40 is mounted on the distal end portion 36. In addition, the hood mounting control is control different from the non-hood-mounting control. The non-hood-mounting control and the hood mounting control are performed based on a result of comparing a light measurement value of the frame 46 with a target light measurement value. A degree of increase in brightness of the frame 46 obtained temporally later the frame 46 from which the light measurement value is obtained (for example, the frame 46 obtained after a predetermined number of frames determined in advance in a range of one frame to several hundred frames from the frame 46 from which the light measurement value is obtained) is greater in the hood mounting control than in the non-hood-mounting control. Here, the frame 46 from which the light measurement value is obtained is an example of a “first image” according to the present disclosure, and the frame 46 obtained temporally later than the frame 46 from which the light measurement value is obtained is an example of a “second image” according to the present disclosure. Hereinafter, for convenience of description, the frame 46 in the former case is referred to as a first frame, and the frame 46 in the latter case is also referred to as a second frame. It should be noted that the non-hood-mounting control and the hood mounting control in the present embodiment are examples of “brightness control” according to the present disclosure. In addition, the non-hood-mounting control in the present embodiment is an example of “first brightness control” according to the present disclosure. In addition, the hood mounting control in the present embodiment is an example of “second brightness control” according to the present disclosure.

First, the non-hood-mounting control will be described. The non-hood-mounting control is control of setting the brightness of the frame 46 (for example, the second frame) obtained by imaging the inside of the large intestine 24 with the camera 30 in a state in which the inside of the large intestine 24 is irradiated with the light 34 in a case in which the hood 40 is not mounted on the distal end portion 36, to first brightness B1. The first brightness B1 is ideal brightness in a state in which the hood 40 is not mounted on the distal end portion 36. The first brightness B1 is determined based on the brightness of the frame 46. Here, the brightness of the frame 46 is an example of a “light measurement value” according to the present disclosure.

In the non-hood-mounting control, the controller 54B acquires the brightness of each pixel of the frame 46 obtained by imaging the inside of the large intestine 24 with the camera 30 in a state in which the inside of the large intestine 24 is irradiated with the light 34 in a case in which the hood 40 is not mounted on the distal end portion 36, from the frame 46 (for example, the first frame). The controller 54B creates a brightness histogram 73 that is a histogram of the brightness acquired from the frame 46. Then, the controller 54B calculates average brightness 70 and peak brightness 72 based on the brightness histogram 73. The average brightness 70 is brightness corresponding to brightness obtained by performing so-called average light measurement on the frame 46 (for example, the first frame). The peak brightness 72 is brightness corresponding to brightness obtained by performing so-called peak light measurement on the frame 46 (for example, the first frame). It should be noted that the average light measurement and the peak light measurement in the present embodiment are examples of “a plurality of light measurement methods” according to the present disclosure. In addition, the average brightness 70 in the present embodiment is an example of an “average light measurement value” according to the present disclosure. In addition, the average brightness 70 and the peak brightness 72 in the present embodiment are examples of a “plurality of light measurement values” according to the present disclosure.

In the example shown in FIG. 6, the controller 54B calculates the average brightness of the entire frame 46 from the brightness histogram 73, as the average brightness 70. The average brightness 70 is brightness representing the overall brightness of the frame 46. Here, the average brightness 70 is shown as an example, but this is merely an example, and for example, brightness representing the overall brightness of the frame 46, such as a median value of the brightness of all the pixels of the frame 46, need only be used.

In addition, in the example shown in FIG. 6, the controller 54B calculates, as the peak brightness 72, the average brightness of a plurality of pixels indicating the hood 40 shown in the frame 46 from a high-brightness region 73A of the brightness histogram 73. Examples of the peak brightness 72 include brightness at which the presence of the hood 40 in the frame 46 can be specified in a case in which the hood 40 is shown in the frame 46.

Here, the high-brightness region 73A refers to a region of brightness (for example, a brightness region of the top 10%) at which the presence of the hood 40 can be specified in the brightness histogram 73 in a case in which the hood 40 is shown in the frame 46. The high-brightness region 73A is determined in advance as a region of the brightness at which the presence of the hood 40 can be specified, by a test using a real machine and/or a computer simulation.

It should be noted that, here, since the color of the hood 40 is transparent, the form example has been described in which the peak brightness 72 is calculated from the high-brightness region 73A, but the present disclosure is not limited thereto. For example, in a case in which the color of the hood 40 is a color other than a transparent color, the region of the brightness at which the presence of the hood 40 can be specified in the brightness histogram 73 is a region of the brightness corresponding to the color of the hood 40. Therefore, the average brightness of the plurality of pixels showing the features of the hood 40 shown in the frame 46 need only be calculated from the region of the brightness corresponding to the color of the hood 40 in the brightness histogram 73.

The controller 54B controls the light source device 20 based on the average brightness 70 and the peak brightness 72, to set the brightness of the frame 46 (for example, the second frame) to the first brightness B1. The first brightness B1 is determined by adjusting the average brightness 70 with respect to the target average brightness 74, and adjusting the peak brightness 72 with respect to the target peak brightness 76. Here, the adjustment of the average brightness 70 with respect to the target average brightness 74 means adjustment of bringing the average brightness 70 closer to the target average brightness 74 (in other words, adjustment of matching the average brightness 70 to the target average brightness 74), and the adjustment of the peak brightness 72 with respect to the target peak brightness 76 means adjustment of bringing the peak brightness 72 closer to the target peak brightness 76 (in other words, adjustment of matching the peak brightness 72 to the target peak brightness 76). For example, the controller 54B controls the light source device 20 such that the average brightness 70 is brought closer to the target average brightness 74 and the peak brightness 72 is brought closer to the target peak brightness 76, thereby setting the brightness of the frame 46 to the first brightness B1.

For example, a ratio between a degree in which the average brightness 70 is brought closer to the target average brightness 74 (in other words, a degree in which the average brightness 70 is made to match the target average brightness 74) and a degree in which the peak brightness 72 is brought closer to the target peak brightness 76 (in other words, a degree in which the peak brightness 72 is made to match the target peak brightness 76) is 1:1. Here, 1:1 means that the degree in which the average brightness 70 is brought closer to the target average brightness 74 and the degree in which the peak brightness 72 is brought closer to the target peak brightness 76 are the same (that is, the average brightness 70 is increased by 50% and the peak brightness 72 is increased by 50% of the current average brightness 70). As described above, the ratio between the degree in which the average brightness 70 is brought closer to the target average brightness 74 and the degree in which the peak brightness 72 is brought closer to the target peak brightness 76 is set to 1:1, so that the shape of the brightness histogram 73 is maintained before and after the adjustment of the brightness of the frame 46. It should be noted that 1:1 is merely an example, and 2:1, 1:0, or 0:0 may be used depending on the preference of the doctor 12 or the like.

The target average brightness 74 may be brightness designated in advance by the doctor 12 or the like, or may be a brightness determined in advance as ideal brightness for matching the average brightness 70 by a test using a real machine and/or a computer simulation. The same applies to the target peak brightness 76. The target average brightness 74 and/or the target peak brightness 76 may be fixed values or may be variable values that are changed in accordance with a given instruction and/or various conditions.

Next, the hood mounting control is mounted will be described. The hood mounting control is control of setting the brightness of the frame 46 (for example, the second frame) obtained by imaging the inside of the large intestine 24 with the camera 30 in a state in which the inside of the large intestine 24 is irradiated with the light 34 in a case in which the hood 40 is mounted on the distal end portion 36, to second brightness B2. The second brightness B2 is ideal brightness in a state in which the hood 40 is mounted on the distal end portion 36. Further, the second brightness B2 is brightness equal to or higher than the first brightness B1. The second brightness B2 is determined based on the brightness of the frame 46.

In addition, the hood mounting control is also control of increasing the average brightness 78 as compared with the average brightness 70 controlled by the non-hood-mounting control.

In the hood mounting control, the controller 54B acquires the brightness of each pixel of the frame 46 obtained by imaging the inside of the large intestine 24 with the camera 30 in a state in which the inside of the large intestine 24 is irradiated with the light 34 in a case in which the hood 40 is mounted on the distal end portion 36, from the frame 46 (for example, the first frame). The controller 54B creates a brightness histogram 81 that is a histogram of the brightness acquired from the frame 46. Then, the controller 54B calculates average brightness 78 and peak brightness 80 based on the brightness histogram 81. The significance of the average brightness 78 is the same as the significance of the average brightness 70, and the significance of the peak brightness 80 is the same as the significance of the peak brightness 72. The calculation of the average brightness 78 and the peak brightness 80 are performed in the same manner as the calculation of the average brightness 70 and the peak brightness 72. In addition, in the example shown in FIG. 6, the significance of a high-brightness region 81A in the brightness histogram 81 is the same as the significance of the high-brightness region 73A in the brightness histogram 73. It should be noted that the average brightness 78 in the present embodiment is an example of an “average light measurement value” according to the present disclosure. Further, in the present embodiment, the average brightness 78 and the peak brightness 80 are examples of “a plurality of light measurement values” according to the present disclosure.

The controller 54B sets the brightness of the frame 46 (for example, the second frame) to the second brightness B2 by executing image processing for brightness adjustment (hereinafter, also simply referred to as “image processing”) based on the average brightness 78 and the peak brightness 80. Examples of the image processing include adjustment of a digital gain. Further, specific examples of the image processing include gamma correction in addition to the adjustment of the digital gain. The second brightness B2 is determined by adjusting the average brightness 78 with respect to the target average brightness 82, and adjusting the peak brightness 80 with respect to the target peak brightness 84. Here, the adjustment of the average brightness 78 with respect to the target average brightness 82 means adjustment of bringing the average brightness 78 closer to the target average brightness 82 (in other words, adjustment of matching the average brightness 78 to the target average brightness 82), and the adjustment of the peak brightness 80 with respect to the target peak brightness 84 means adjustment of bringing the peak brightness 80 closer to the target peak brightness 84 (in other words, adjustment of matching the peak brightness 80 to the target peak brightness 84).

For example, the controller 54B executes the image processing such that the average brightness 78 is brought closer to the target average brightness 82 and the peak brightness 80 is brought closer to the target peak brightness 84. In addition, the controller 54B executes the image processing such that a ratio of the degree in which the peak brightness 80 is brought closer to the target peak brightness 84 to the degree in which the average brightness 78 is brought closer to the target average brightness 82 is greater than a ratio of the degree in which the peak brightness 72 is brought closer to the target peak brightness 76 to the degree in which the average brightness 70 is brought closer to the target average brightness 74. In addition, the controller 54B executes the image processing such that the average brightness 78 is higher than the average brightness 70 after the adjustment in the non-hood-mounting control. The image processing executed herein is processing of adjusting the brightness of each pixel of the frame 46.

The degree in which the average brightness 78 is brought closer to the target average brightness 82 is greater than the degree in which the peak brightness 80 is brought closer to the target peak brightness 84. For example, the ratio between the degree in which the average brightness 78 is brought closer to the target average brightness 82 and the degree in which the peak brightness 80 is brought closer to the target peak brightness 84 is 3:1. Here, 3:1 means that the degree in which the average brightness 78 is brought closer to the target average brightness 82 is three times the degree in which the peak brightness 80 is brought closer to the target peak brightness 84.

As described above, by setting the ratio between the degree in which the average brightness 78 is brought closer to the target average brightness 82 and the degree in which the peak brightness 80 is brought closer to the target peak brightness 84 to 3:1, a difference between the high-brightness region 81A and the region other than the high-brightness region 81A in the brightness histogram 81 after the adjustment of the brightness of the frame 46 is less than a difference between the high-brightness region 81A and the region other than the high-brightness region 81A in the brightness histogram 81 before the adjustment of the brightness of the frame 46. It should be noted that 3:1 is merely an example, and 2:1 or 1:0 may be used depending on the preference of the doctor 12 or the like. In any case, a degree of use of the average brightness 70 and the peak brightness 72 in the non-hood-mounting control is different from a degree of use of the average brightness 78 and the peak brightness 80 in the hood mounting control. For example, the degree of use of the average brightness 78 in the hood mounting control is greater than the degree of use of the average brightness 70 in the non-hood-mounting control.

The target average brightness 82 may be brightness designated in advance by the doctor 12 or the like, or may be a brightness determined in advance as ideal brightness for matching the average brightness 78 by a test using a real machine and/or a computer simulation. The same applies to the target peak brightness 84. The target average brightness 82 and/or the target peak brightness 84 may be fixed values or may be variable values that are changed in accordance with a given instruction and/or various conditions.

It should be noted that, in the present embodiment, although the form example has been described in which the brightness of the frame 46 (for example, the second frame) is increased by setting a degree of an influence of the image processing for brightness adjustment on the frame 46 (for example, the second frame) in a case in which the image processing is performed on the frame 46 (for example, the second frame) to be higher in the hood mounting control than in the non-hood-mounting control (for example, increasing the digital gain), this is merely an example. For example, in order to increase the brightness of the frame 46 (for example, the second frame), the exposure time with the endoscope 14 (that is, the exposure time with the image sensor 64) in a case in which the imaging for obtaining the frame 46 (for example, the second frame) is performed may be lengthened, or the light quantity of the light 34 in a case in which the imaging for obtaining the frame 46 (for example, the second frame) is performed may be increased. In addition, the adjustment of the exposure time, the adjustment of the light quantity of the light 34, and the image processing (for example, the adjustment of the digital gain) may be sequentially performed. For example, the exposure time is allowed up to a certain time (for example, 1/30 seconds or 1/60 seconds), and in a case in which an exposure time longer than a certain time is required, the influence of the shake of the image is remarkable, and thus the light quantity of the light 34 need only be adjusted. In the adjustment of the digital gain, there is a concern that noise may increase, and thus the adjustment of the digital gain may be used in a case in which the brightness of the frame 46 cannot be sufficiently increased by adjusting the exposure time and adjusting the light quantity of the light 34.

Next, the operations and effects of the portions of the endoscope system 10 according to the present embodiment that are related to the present disclosure will be described.

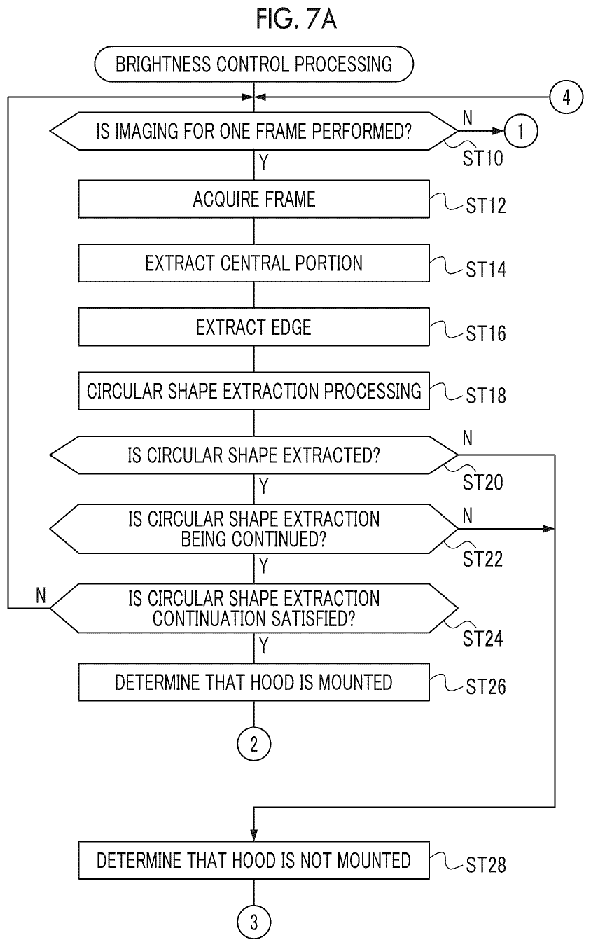

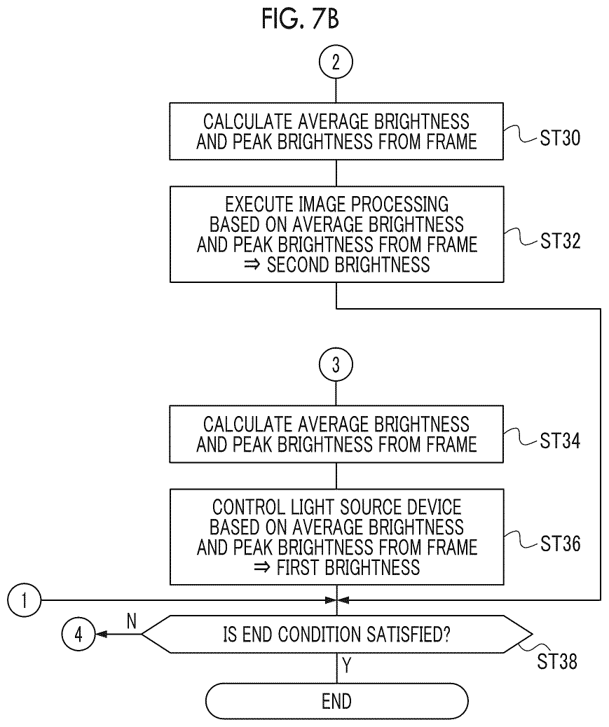

FIGS. 7A and 7B show an example of a flow of the brightness control processing executed by the processor 54. The flowcharts shown in FIGS. 7A and 7B are examples of an “operation method of a control device” according to the present disclosure. Here, for convenience of description, a case will be described in which the brightness control processing is executed by the processor 54 in a case in which the inside of the large intestine 24 is irradiated with the light 34 in a state in which the distal end portion 36 is inserted into the large intestine 24.

In the brightness control processing shown in FIG. 7A, in step ST10, the determination unit 54A determines whether or not the imaging for one frame is performed by the camera 30. In step ST10, in a case in which the imaging for one frame is not performed by the camera 30, a negative determination is made, and the brightness control processing proceeds to step ST38 shown in FIG. 7B. In step ST10, in a case in which the imaging for one frame is performed by the camera 30, a positive determination is made, and the brightness control processing proceeds to step ST12.

In step ST12, the determination unit 54A acquires the frame 46 from the camera 30 (see FIG. 5). After the processing of step ST12 is executed, the brightness control processing proceeds to step ST14.

In step ST14, the determination unit 54A extracts the central portion 46A from the frame 46 (see FIG. 5). After the processing of step ST14 is executed, the brightness control processing proceeds to step ST16.

In step ST16, the determination unit 54A extracts the edge 46A1 from the central portion 46A (see FIG. 5). After the processing of step ST16 is executed, the brightness control processing proceeds to step ST18.

In step ST18, the determination unit 54A executes the circular shape extraction processing using the edge 46A1 (see FIG. 5). After the processing of step ST18 is executed, the brightness control processing proceeds to step ST20.

In step ST20, the determination unit 54A determines whether or not the circular shape 46A2 is extracted by executing the circular shape extraction processing (see FIG. 5). In step ST20, in a case in which the circular shape 46A2 is not extracted by executing the circular shape extraction processing, a negative determination is made, and the brightness control processing proceeds to step ST28. In step ST20, in a case in which the circular shape 46A2 is extracted by executing the circular shape extraction processing, a positive determination is made, and the brightness control processing proceeds to step ST22.

In step ST22, the determination unit 54A determines whether or not the extraction of the circular shape 46A2 is being continued over the plurality of frames 46 in time series (see FIG. 5). In step ST22, in a case in which the extraction of the circular shape 46A2 is not being continued over the plurality of frames 46 in time series, a negative determination is made, and the brightness control processing proceeds to step ST28. In step ST22, in a case in which the extraction of the circular shape 46A2 is being continued over the plurality of frames 46 in time series, a positive determination is made, and the brightness control processing proceeds to step ST24.

In step ST24, the determination unit 54A determines whether or not the circular shape extraction continuation condition is satisfied (see FIG. 5). In step ST24, in a case in which the circular shape extraction continuation condition is not satisfied, a negative determination is made, and the brightness control processing proceeds to step ST10. In step ST24, in a case in which the circular shape extraction continuation condition is satisfied, a positive determination is made, and the brightness control processing proceeds to step ST26.

In step ST26, the determination unit 54A determines that the hood 40 is mounted on the distal end portion 36 (see FIG. 5). After the processing of step ST26 is executed, the brightness control processing proceeds to step ST30 shown in FIG. 7B.

In step ST28, the determination unit 54A determines that the hood 40 is not mounted on the distal end portion 36 (see FIG. 5). After the processing of step ST28 is executed, the brightness control processing proceeds to step ST34 shown in FIG. 7B.

In step ST30 shown in FIG. 7B, the controller 54B calculates the average brightness 78 and the peak brightness 80 from the frame 46 acquired in step ST12 (see FIG. 6). After the processing of step ST30 is executed, the brightness control processing proceeds to step ST32.

In step ST32, the controller 54B executes the image processing on the frame 46 acquired in step ST12 based on the average brightness 78 and the peak brightness 80, to set the brightness of the frame 46 to the second brightness B2 (see FIG. 6). After the processing of step ST32 is executed, the brightness control processing proceeds to step ST38.

In step ST34, the controller 54B calculates the average brightness 70 and the peak brightness 72 from the frame 46 acquired in step ST12 (see FIG. 6). After the processing of step ST34 is executed, the brightness control processing proceeds to step ST36.

In step ST36, the controller 54B controls the light source device 20 to set the brightness of the frame 46 acquired in step ST12 to the first brightness B1 (see FIG. 6).

It should be noted that, in step ST36, although the form example has been described in which the brightness of the frame 46 is set to the first brightness B1 by controlling the light source device 20, this is merely an example, and the brightness of the frame 46 may be set to the first brightness B1 by executing image processing (for example, processing of adjusting the brightness of each pixel of the frame 46) on the frame 46. After the processing of step ST36 is executed, the brightness control processing proceeds to step ST38.

In step ST38, the controller 54B determines whether or not a brightness control processing end condition is satisfied. Examples of the brightness control processing end condition include a condition in which an instruction to end the brightness control processing is given to the endoscope system 10 (for example, a condition that the reception device 62 receives an instruction to end the brightness control processing).

In step ST38, in a case in which the brightness control processing end condition is not satisfied, a negative determination is made, and the brightness control processing proceeds to step ST10 shown in FIG. 7A. In step ST38, in a case in which the brightness control processing end condition is satisfied, a positive determination is made, and the brightness control processing ends.

As described above, in the endoscope system 10 according to the present embodiment, the non-hood-mounting control and the hood mounting control are performed as the brightness control of bringing the brightness of the frame 46 closer to the target brightness. The non-hood-mounting control is performed in a case in which the hood 40 is not mounted on the distal end portion 36, and the hood mounting control is performed in a case in which the hood 40 is mounted on the distal end portion 36. In addition, the hood mounting control is control different from the non-hood-mounting control. Accordingly, it is possible to prevent the frame 46 (for example, the second frame) from becoming dark overall due to the brightness control (for example, the non-hood-mounting control) that is performed due to the frame 46 (for example, the first frame) becoming bright due to the influence of the halation that occurs in a case in which the hood 40 is mounted on the distal end portion 36.

In addition, in the endoscope system 10 according to the present embodiment, the non-hood-mounting control is performed based on a result of comparing the light measurement value (for example, the average brightness 70 and the peak brightness 72) of the frame 46 (for example, the first frame) with the target light measurement value (for example, the target average brightness 74 and the target peak brightness 76), and the hood mounting control is performed based on a result of comparing the light measurement value (for example, the average brightness 78 and the peak brightness 80) of the frame 46 (for example, the first frame) with the target light measurement value (for example, the target average brightness 82 and the target peak brightness 84). The degree of increase in the brightness of the frame 46 (for example, the second frame) is greater in the hood mounting control than in the non-hood-mounting control. Even in a case in which the hood 40 is mounted on the distal end portion 36, in a case in which the control similar to the non-hood-mounting control is performed, the control of preventing the frame 46 from becoming bright due to the influence of the halation that occurs in the hood 40, and thus the frame 46 becomes dark overall. However, in the present embodiment, by setting the degree of increase in the brightness of the frame 46 (for example, the second frame) in the hood mounting control to be greater than the degree of increase in the brightness of the frame 46 (for example, the second frame) in the non-hood-mounting control, it is possible to obtain the frame 46 that is brighter overall than the frame 46 (for example, the second frame) in which the overall brightness is adjusted by the brightness control (for example, the non-hood-mounting control) that is performed due to the frame 46 (for example, the first frame) becoming bright due to the influence of the halation that occurs in a case in which the hood 40 is mounted on the distal end portion 36.

In addition, in the endoscope system 10 according to the present embodiment, the brightness of the frame 46 (for example, the second frame) is increased by lengthening the exposure time with the endoscope 14 in a case in which the imaging for obtaining the frame 46 (for example, the second frame) is performed, increasing the light quantity of the light 34 in a case in which the imaging for obtaining the frame 46 (for example, the second frame) is performed, and increasing the degree of the influence of the image processing for brightness adjustment on the frame 46 (for example, the second frame) in a case in which the image processing is performed on the frame 46 (for example, the second frame). Accordingly, it is possible to obtain the frame 46 that is brighter than the frame 46 in which the overall brightness is adjusted by the brightness control (for example, the non-hood-mounting control) that is performed due to the frame 46 (for example, the first frame) becoming bright due to the influence of the halation that occurs in a case in which the hood 40 is mounted on the distal end portion 36.

In addition, in the present embodiment, the degrees of use of the average brightness 70 and the peak brightness 72 in the non-hood-mounting control are 1:1, while the degrees of use of the average brightness 78 and the peak brightness 80 in the hood mounting control are 3:1. As described above, the degree of use of the average brightness 78 in the hood mounting control is greater than the degree of use of the average brightness 70 in the non-hood-mounting control. As described above, it is possible to prevent the frame 46 (for example, the second frame) from becoming dark overall due to the brightness control (for example, the non-hood-mounting control) that is performed due to the frame 46 (for example, the first frame) becoming bright due to the influence of the halation that occurs in a case in which the hood 40 is mounted on the distal end portion 36.

In addition, in the endoscope system 10 according to the present embodiment, in a case in which the hood 40 is not mounted on the distal end portion 36, the brightness of the frame 46 is set to the first brightness B1. On the other hand, in a case in which the hood 40 is mounted on the distal end portion 36, the brightness of the frame 46 is set to the second brightness B2. The first brightness B1 is ideal brightness in a state in which the hood 40 is not mounted on the distal end portion 36, and the second brightness B2 is ideal brightness in a state in which the hood 40 is mounted on the distal end portion 36. Therefore, the brightness of the frame 46 can be set to the ideal brightness depending on whether the hood 40 is not mounted on the distal end portion 36 or the hood 40 is mounted on the distal end portion 36.

In addition, in the endoscope system 10 according to the present embodiment, whether or not the hood 40 is mounted on the distal end portion 36 is specified based on the circular shape 46A2 extracted from the frame 46 and the circular shape extraction continuation condition. That is, whether or not the hood 40 is mounted on the distal end portion 36 is specified in accordance with whether or not the extraction of the circular shape 46A2 from the frame 46 is continuously performed for the predetermined time or longer over the plurality of frames 46. Therefore, it is possible to specify whether or not the hood 40 is mounted on the distal end portion 36 without specifying whether or not the hood 40 is mounted on the distal end portion 36 based on a visual inspection result of the doctor 12.

In addition, in the endoscope system 10 according to the present embodiment, the circular shape 46A2 used for specifying whether or not the hood 40 is mounted on the distal end portion 36 is extracted from the central portion 46A of the frame 46. Therefore, it is possible to reduce the computational load required for extracting the circular shape 46A2 used for specifying whether or not the hood 40 is mounted on the distal end portion 36, as compared with a case in which the circular shape 46A2 used for specifying whether or not the hood 40 is mounted on the distal end portion 36 is extracted from the entire frame 46.

In addition, in the endoscope system 10 according to the present embodiment, the second brightness B2 of the frame 46 in a case in which the hood 40 is mounted on the distal end portion 36 is set to be equal to or higher than the first brightness B1 of the frame 46 in a case in which the hood 40 is not mounted on the distal end portion 36. Therefore, even in a case in which the hood 40 is mounted on the distal end portion 36, the brightness of the frame 46 can be set to be equal to or higher than the brightness in a case in which the hood 40 is not mounted on the distal end portion 36.

In addition, in the endoscope system 10 according to the present embodiment, the second brightness B2, which is the brightness of the frame 46 in a case in which the hood 40 is mounted on the distal end portion 36, is determined based on the brightness (the average brightness 78 and the peak brightness 80 in the example shown in FIG. 6) of the frame 46. In addition, the first brightness B1, which is the brightness of the frame 46 in a case in which the hood 40 is not mounted on the distal end portion 36, is also determined based on the brightness (the average brightness 70 and the peak brightness 72 in the example shown in FIG. 6) of the frame 46. Therefore, the first brightness B1 and the second brightness B2 can be set to appropriate brightness with high accuracy, as compared with a case in which the first brightness B1 and the second brightness B2 are determined only by elements that are completely irrelevant to the brightness of the frame 46 among all elements constituting the frame 46.

In addition, in the endoscope system 10 according to the present embodiment, the control of increasing the average brightness 78 for setting the brightness of the frame 46 to the second brightness B2 in a case in which the hood 40 is mounted on the distal end portion 36 is set to control of making the average brightness 78 to be higher as compared with the control of increasing the average brightness 70 for setting the brightness of the frame 46 to the first brightness B1 in a case in which the hood 40 is not mounted on the distal end portion 36. That is, the degree of increase in the average brightness 78 is greater than the degree of increase in the average brightness 70. Accordingly, in a case in which the hood 40 is mounted on the distal end portion 36, the brightness of the observation target to be observed by the doctor 12 via the frame 46 can be set to appropriate brightness.

In the endoscope system 10 according to the present embodiment, the second brightness B2 is determined by adjusting the average brightness 78 with respect to the target average brightness 82 and adjusting the peak brightness 80 with respect to the target peak brightness 84. Accordingly, the second brightness B2 can be set to appropriate brightness, as compared with a case in which the second brightness B2 is determined only by adjusting the average brightness 78 with respect to the target average brightness 82 or the second brightness B2 is determined only by adjusting the peak brightness 80 with respect to the target peak brightness 84.

In the endoscope system 10 according to the present embodiment, as the adjustment of the average brightness 78 with respect to the target average brightness 82, the average brightness 78 is adjusted to match the target average brightness 82. Further, as the adjustment of the peak brightness 80 with respect to the target peak brightness 84, the peak brightness 80 is adjusted to match the target peak brightness 84. Here, a degree in which the average brightness 78 is made to match the target average brightness 82 is greater than a degree in which the peak brightness 80 is made to match the target peak brightness 84. Therefore, it is possible to easily set the second brightness B2 to appropriate brightness as compared with a case in which the degree in which the peak brightness 80 is made to match the target peak brightness 84 is greater than the degree in which the average brightness 78 is made to match the target average brightness 82.

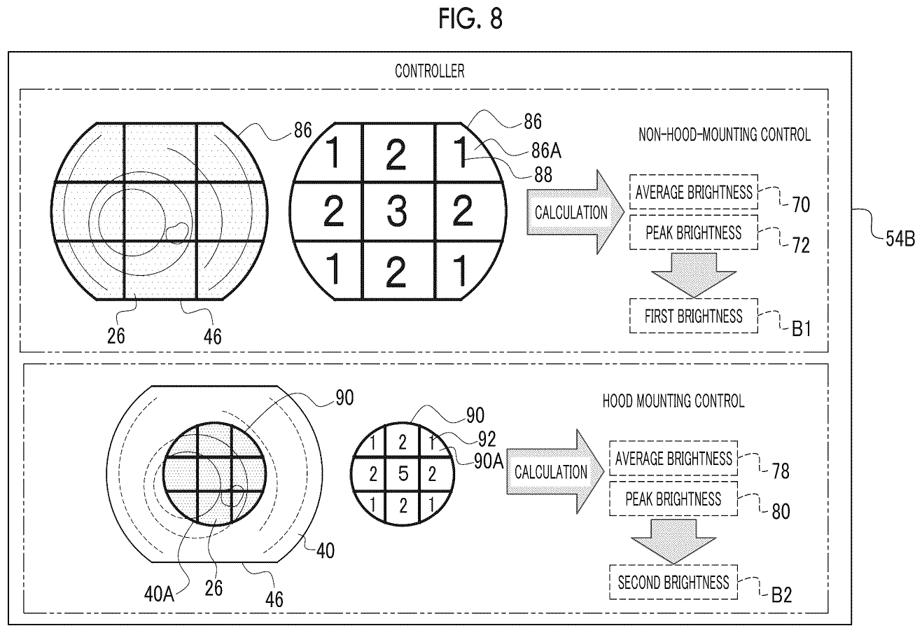

It should be noted that, in the above-described embodiment, the form example has been described in which the average brightness 70, the average brightness 78, the peak brightness 72, and the peak brightness 80 are calculated using the brightness of all the pixels included in the frame 46, but the present disclosure is not limited thereto. For example, as shown in FIG. 8, the average brightness 70 and the peak brightness 72 used for obtaining the first brightness B1 may be determined based on the brightness of a first range 86 of the frame 46, and the average brightness 78 and the peak brightness 80 used for obtaining the second brightness B2 may be determined based on the brightness of a second range 90 that is a range closer to the center of the frame 46 than the first range 86 (in other words, an inner region of the frame 46 with respect to the first range 86). The second range 90 is an example of a “specific region” according to the present disclosure. The second range 90 is a specific region determined in accordance with the hood 40. That is, the geometrical characteristics of the second range 90 are not constant and are determined in accordance with the shape and/or size of the hood 40. Accordingly, it is possible to reduce the computational load required for calculating the average brightness 78 and the peak brightness 80 (in other words, it is possible to reduce the computational load required for obtaining the second brightness B2) as compared with a case in which the second brightness B2 is determined based on the brightness within the same range of the frame 46 as in a case in which the first brightness B1 is determined (the brightness within the first range 86 in the example shown in FIG. 8).

In the example shown in FIG. 8, a closed region corresponding to the opening 40A shown in the frame 46 is shown as an example of the second range 90, but the present disclosure is not limited thereto, and the second range 90 need only be a range (that is, the closed region) closer to the center than the first range 86.

In addition, in the example shown in FIG. 8, the entire range of the frame 46 is shown as the first range 86, but this is merely an example. For example, a range wider than the second range 90 and narrower than the entire range of the frame 46 may be used, and in this case, the average brightness 70 and the peak brightness 72 are calculated based on the brightness of each pixel included in the closed region narrower than the entire range of the frame 46.

In the example shown in FIG. 8, the first range 86 includes a plurality of first divided regions 86A. The plurality of first divided regions 86A are obtained by dividing the first range 86. In the example shown in FIG. 8, nine (=3×3) divided regions are shown as examples of the plurality of first divided regions 86A. In general, since the degree of attention by the doctor 12 is higher as the position is closer to the center of the frame 46, a larger first weight 88 is given to the plurality of first divided regions 86A as the position is closer to the center of the frame 46. The first brightness B1 is determined based on the first weight 88, which is given to each of the plurality of first divided regions 86A, and the frequency of the brightness of the first range 86.

That is, the brightness of each pixel in the first range 86 is adjusted by being multiplied by the corresponding first weight 88 (that is, the first weight 88 given to the first divided region 86A to which the pixel belongs), and then the average brightness 70 and the peak brightness 72 are calculated based on the adjusted brightness and the frequency of the adjusted brightness. The average brightness 70 and the peak brightness 72 are obtained for each first divided region 86A, and have a size corresponding to the first divided region 86A. The brightness of the frame 46 is adjusted by executing the image processing of adjusting the brightness of each pixel included in the first range 86 with respect to the frame 46 based on the calculated average brightness 70 and peak brightness 72, and as a result, the brightness of the frame 46 is set to the first brightness B1. In this way, since the first brightness B1 is determined with higher importance as the position is closer to the center of the frame 46, in a case in which the hood 40 is not mounted on the distal end portion 36, it is possible to provide the doctor 12 with the frame 46 having good visibility of a portion with a high degree of attention by the doctor 12.

In the example shown in FIG. 8, the second range 90 includes a plurality of second divided regions 90A. The plurality of second divided regions 90A are obtained by dividing the frame 46 by a division method different from the non-hood-mounting control. The division method is determined in accordance with the instrument mounted on the distal end portion 36. Here, since the hood 40 is mounted on the distal end portion 36, the division method is determined in accordance with the hood 40. The plurality of second divided regions 90A are obtained by dividing the second range 90 using the division method in accordance with the hood 40. In the example shown in FIG. 8, nine (=3×3) divided regions are shown as examples of the plurality of second divided regions 90A. In general, since the degree of attention by the doctor 12 is higher as the position is closer to the center of the frame 46, a greater second weight 92 is given to the plurality of second divided regions 90A as the position is closer to the center of the frame 46. The second brightness B2 is determined based on the second weight 92, which is given to each of the plurality of second divided regions 90A, and the frequency of the brightness of the second range 90.

That is, the brightness of each pixel in the second range 90 is adjusted by being multiplied by the corresponding second weight 92 (that is, the second weight 92 given to the second divided region 90A to which the pixel belongs), and then the average brightness 78 and the peak brightness 80 are calculated based on the adjusted brightness and the frequency of the adjusted brightness. The average brightness 78 and the peak brightness 80 are obtained for each second divided region 90A, and have a size corresponding to the second divided region 90A. The brightness of the frame 46 is adjusted by executing the image processing of adjusting the brightness of each pixel included in the second range 90 with respect to the frame 46 based on the calculated average brightness 78 and peak brightness 80, and as a result, the brightness of the frame 46 is set to the second brightness B2. In this way, since the second brightness B2 is determined with higher importance as the position is closer to the center of the frame 46, in a case in which the hood 40 is mounted on the distal end portion 36, it is possible to provide the doctor 12 with the frame 46 having good visibility of a portion with a high degree of attention by the doctor 12.

In the example shown in FIG. 8, a degree in which the second weight 92 is increased as the position is closer to the center of the frame 46 (for example, a degree in which the second weight 92 is increased as the position is closer to the center of the second range 90) is greater than a degree in which the first weight 88 is increased as the position is closer to the center of the frame 46 (for example, a degree in which the first weight 88 is increased as the position is closer to the center of the first range 86). In this way, the brightness of the frame 46 is determined with higher importance as the position is closer to the center of the frame 46 in a case in which the hood 40 is mounted on the distal end portion 36 than in a case in which the hood 40 is not mounted on the distal end portion 36. As a result, in a case in which the hood 40 is mounted on the distal end portion 36, it is possible to provide the doctor 12 with the frame 46 having good visibility of a portion with a high degree of attention by the doctor 12.

In the example shown in FIG. 8, the nine divided regions are shown as an example of the plurality of first divided regions 86A, but this is merely an example, and the number of first divided regions 86A may be less than nine or may be ten or more. In addition, in the example shown in FIG. 8, the nine divided regions are shown as an example of the plurality of second divided regions 90A, but this is merely an example, and the number of second divided regions 90A may be less than nine or may be ten or more.

In the example shown in FIG. 8, the form example has been described in which the brightness is adjusted by the first weight 88 and the second weight 92, but this is merely an example, and a pixel value having magnitude determined in accordance with the first divided region 86A may be used, or a pixel value having magnitude determined in accordance with the second divided region 90A may be used.

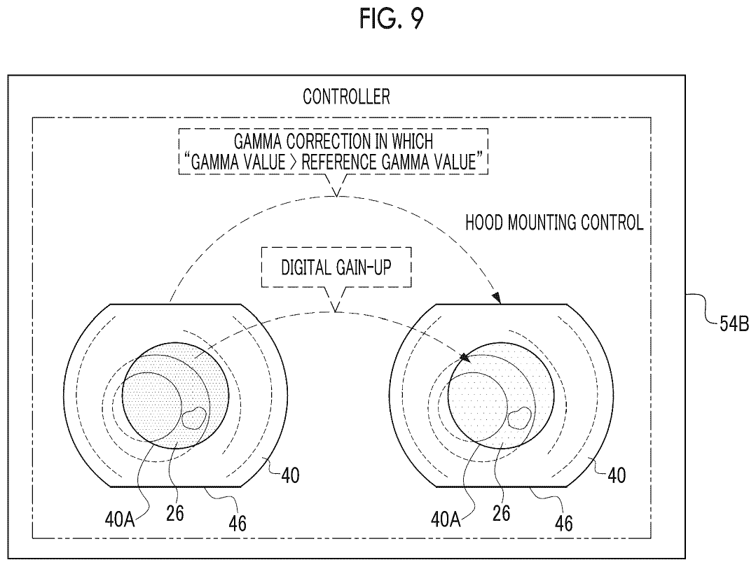

FIG. 9 shows an example of image processing performed to increase the brightness in the hood mounting control. As shown in FIG. 9, in the hood mounting control, image processing including digital gain-up for an inner region of the opening 40A included in the frame 46 is executed in the frame 46. As a result, it is possible to increase the visibility of a region (for example, the inner region of the opening 40A) that generally has the degree of attention by the doctor 12 among all regions of the frame 46 obtained in a case in which the hood 40 is mounted on the distal end portion 36.

In addition, the controller 54B may perform digital gain-up on the inner region of the opening 40A shown in the frame 46 in the frame 46, to set the second brightness B2 to brightness at which the visibility of the inner region of the opening 40A shown in the frame 46 is better than the visibility of an outer region of the opening 40A shown in the frame 46. That is, the controller 54B may set the second brightness B2 to brightness having a feature in which the inner region of the opening 40A shown in the frame 46 is brighter than the outer region of the opening 40A shown in the frame 46. In this way, the visibility of the region (for example, the inner region of the opening 40A) with a higher degree of attention by the doctor 12 can be increased than the visibility of the region (for example, the outer region of the opening 40A) with a lower degree of attention by the doctor 12.

Further, as shown in FIG. 9, in the hood mounting control, image processing including gamma correction of increasing a gamma value to be higher than a reference gamma value (for example, a default gamma value or a gamma value designated by the doctor 12 or the like) is executed. As a result, it is possible to realize an increase in overall brightness of the frame 46 obtained in a case in which the hood 40 is mounted on the distal end portion 36.

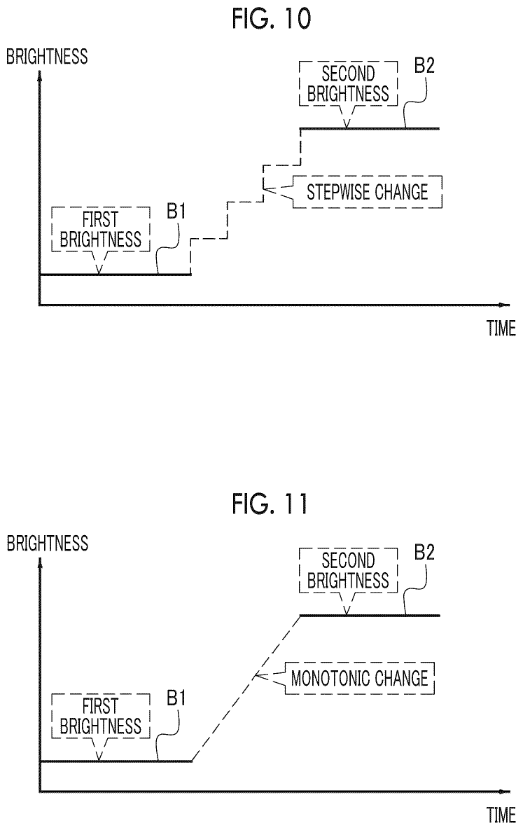

FIGS. 10 and 11 show examples of a switching aspect in a case in which the first brightness B1 is switched to the second brightness B2. In a case in which the first brightness B1 is switched to the second brightness B2, the brightness of the frame 46 may be changed stepwise as shown in FIG. 10. Examples of the stepwise change in the brightness of the frame 46 include a form example in which the brightness of the frame 46 is changed in a plurality of steps at a constant time interval and constant magnitude.

In addition, in a case in which the first brightness B1 is switched to the second brightness B2, the brightness of the frame 46 may be changed monotonically as shown in FIG. 11. Examples of the monotonic change in the brightness of the frame 46 include a form example in which the brightness of the frame 46 is linearly changed and a form example in which the brightness of the frame 46 is exponentially changed.

In the examples shown in FIGS. 10 and 11, although the aspect example is shown in which the first brightness B1 is changed to the second brightness B2, even in a case in which the second brightness B2 is changed to the first brightness B1, the brightness of the frame 46 may be changed stepwise, or the brightness of the frame 46 may be changed monotonically.

The stepwise or monotonic change in the brightness of the frame 46 (for example, the second frame) is achieved by lengthening, stepwise, the exposure time with the endoscope 14, increasing, stepwise, the light quantity of the light 34, or increasing, stepwise, the degree of influence of the image processing for brightness adjustment on the frame 46 in a case in which the image processing is performed on the frame 46.

As described above, in a case in which the first brightness B1 and the second brightness B2 are switched from one to the other, by changing the brightness of the frame 46 stepwise or changing the brightness of the frame 46 monotonically, it is possible to reduce the visual discomfort caused by the change in the brightness of the frame 46, as compared with a case in which the first brightness B1 and the second brightness B2 are directly switched from one to the other.

In the above-described embodiment, although the form example has been described in which whether or not the hood 40 is mounted on the distal end portion 36 is specified based on the circular shape 46A2, which is an example of the feature value of the central portion 46A of the frame 46, and the circular shape extraction continuation condition, the present disclosure is not limited thereto. For example, whether or not the hood 40 is mounted on the distal end portion 36 may be specified based on a feature value related to the brightness in the frame 46 and a condition given to the feature value related to the brightness in the frame 46.

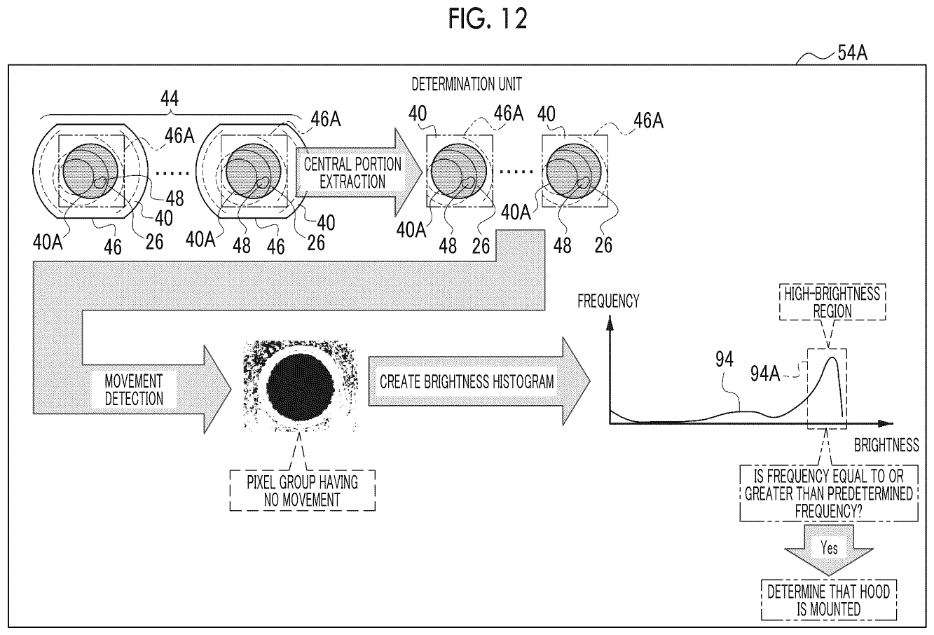

Here, examples of the feature value related to the brightness in the frame 46 include a brightness histogram 94 as shown in FIG. 12. Examples of the condition given to the feature value related to the brightness in the frame 46 include a time condition in which the brightness histogram 94 continues for a predetermined time (for example, a predetermined number of frames) or longer.

The brightness histogram 94 is created by the determination unit 54A based on a pixel group having no movement. The brightness histogram 94 is a histogram of the brightness of the pixel group having no movement. Here, the pixel group having no movement refers to a pixel group in which there is no change in brightness between the plurality of frames 46 (for example, between two frames 46 temporally adjacent in time) among the plurality of frames 46 in time series included in the moving image 44. The brightness histogram 94 includes a high-brightness region 94A that is synonymous with the high-brightness regions 73A and 81A described in the above-described embodiment. The brightness histogram 94 has a feature in which a frequency of the high-brightness region 94A (that is, a frequency of the brightness at which the feature of the hood 40 shown in the frame 46 can be specified) is equal to or higher than a predetermined frequency. Examples of the predetermined frequency include a frequency determined in advance by a test using a real machine and/or a computer simulation as the frequency of the brightness at which the presence of the hood 40 can be specified.

In a case in which the brightness histogram 94 having the same shape is continuously created for the predetermined time or longer, the determination unit 54A determines that the hood 40 is mounted on the distal end portion 36. In addition, in a case in which the brightness histogram 94 having the same shape is not created and in a case in which the creation of the brightness histogram 94 having the same shape is interrupted in a time shorter than the predetermined time, the determination unit 54A determines that the hood 40 is not mounted on the distal end portion 36. In this way, the same effect as the effect of the above-described embodiment can be obtained.

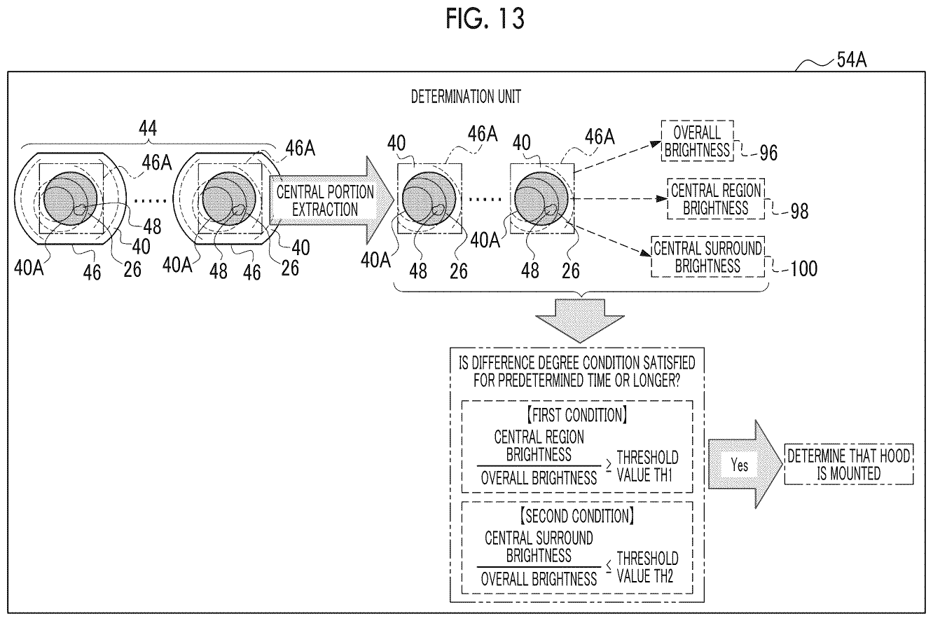

In the example shown in FIG. 12, the form example has been described in which it is determined that the hood 40 is mounted on the distal end portion 36 in a case in which the brightness histogram 94 having the same shape is continuously created for the predetermined time or longer, but the present disclosure is not limited thereto. For example, as shown in FIG. 13, in a case in which a difference degree between overall brightness 96, central region brightness 98, and central surround brightness 100 satisfies a difference degree condition, it may be determined that the hood 40 is mounted on the distal end portion 36.

Here, the overall brightness 96 refers to representative brightness (here, as an example, the average brightness) of all the pixels included in the central portion 46A. In addition, the central region brightness 98 refers to representative brightness (here, as an example, the average brightness) of the central region of the central portion 46A. In addition, the central surround brightness 100 refers to the representative brightness (here, as an example, the average brightness) around the central region of the central portion 46A.

The difference degree condition includes a first condition and a second condition. The first condition refers to a condition in which a ratio of the central region brightness 98 to the overall brightness 96 is equal to or greater than a threshold value TH1. The second condition refers to a condition in which a ratio of the central surround brightness 100 to the overall brightness 96 is equal to or less than a threshold value TH2. Here, the ratio is shown as an example, but a difference may be applied as the difference degree instead of the ratio.

The threshold value TH2 is less than the threshold value TH1. Examples of the threshold value TH1 include a value determined in advance by a test using a real machine and/or a computer simulation as a lower limit value of the ratio of the central region brightness 98 to the overall brightness 96 in a case in which the hood 40 including the opening 40A is shown in the central portion 46A. In addition, examples of the threshold value TH2 include a value that is determined in advance by a test using a real machine and/or a computer simulation as an upper limit value of the ratio of the central surround brightness 100 to the overall brightness 96 in a case in which the hood 40 including the opening 40A is reflected in the central portion 46A.

The threshold value TH2 may be the same as the threshold value TH1. In addition, the threshold value TH1 and/or the threshold value TH2 may be variable values that are changed in accordance with a given instruction and/or various conditions.

In a case in which the central portion 46A that satisfies both the first condition and the second condition is continuously obtained for the predetermined time or longer, it is determined that the hood 40 is mounted on the distal end portion 36. In a case in which the first condition and/or the second condition is not satisfied and in a case in which the acquisition of the central portion 46A that satisfies both the first condition and the second condition is interrupted in a time shorter than the predetermined time, the determination unit 54A determines that the hood 40 is not mounted on the distal end portion 36. In this way, the same effect as the effect of the above-described embodiment can be obtained.

Here, the form example has been described in which the overall brightness 96, the central region brightness 98, and the central surround brightness 100 are obtained from the central portion 46A which is a part of the frame 46, but this is merely an example, and the overall brightness 96, the central region brightness 98, and the central surround brightness 100 may be obtained from the entire frame 46, and the overall brightness 96, the central region brightness 98, and the central surround brightness 100 need only be obtained from a representative region of the frame 46.

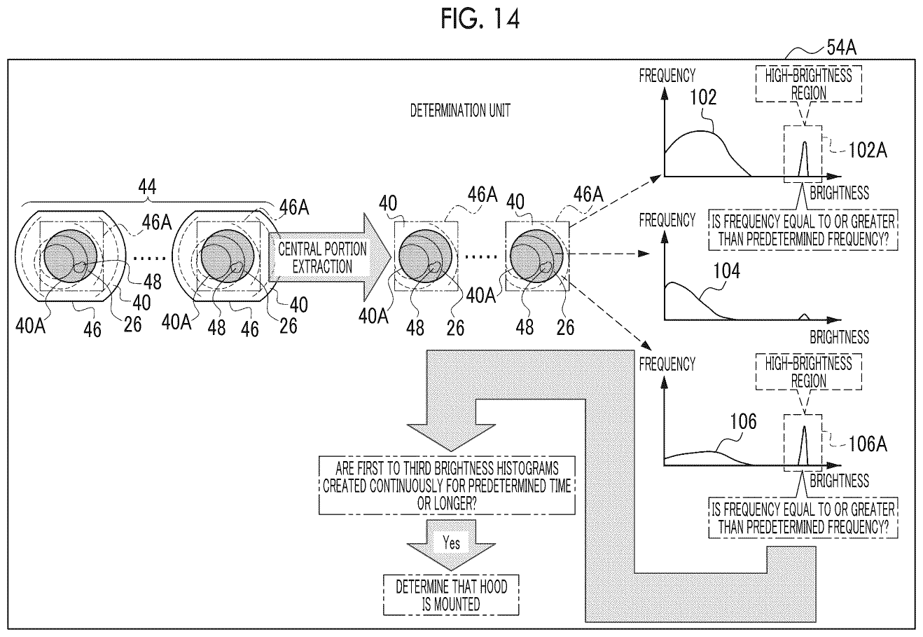

In the example shown in FIG. 13, the form example has been described in which it is determined that the hood 40 is mounted on the distal end portion 36 in a case in which the central portion 46A that satisfies both the first condition and the second condition is continuously obtained for the predetermined time or longer, but the present disclosure is not limited thereto. For example, as shown in FIG. 14, in a case in which a first brightness histogram 102, a second brightness histogram 104, and a third brightness histogram 106 are continuously obtained for the predetermined time or longer (that is, in a case in which the time condition is satisfied), it may be determined that the hood 40 is mounted on the distal end portion 36.

The first brightness histogram 102 is a histogram of the brightness of each pixel included in the entire central portion 46A of the frame 46 obtained by imaging the inside of the large intestine 24 with the camera 30 in a case in which the inside of the large intestine 24 is irradiated with the light 34 in a state in which the hood 40 is mounted on the distal end portion 36. The first brightness histogram 102 includes a high-brightness region 102A that is synonymous with the high-brightness region 94A shown in FIG. 12.

The second brightness histogram 104 is a histogram of the brightness of each pixel included in a central region of the central portion 46A of the frame 46 obtained by imaging the inside of the large intestine 24 by the camera 30 in a case in which the inside of the large intestine 24 is irradiated with the light 34 in a state in which the hood 40 is mounted on the distal end portion 36.

The third brightness histogram 106 is a histogram of the brightness of each pixel included in the surroundings of the central region of the central portion 46A (in other words, a region other than the central region in the central portion 46A) of the frame 46 obtained by imaging the inside of the large intestine 24 with the camera 30 in a case in which the inside of the large intestine 24 is irradiated with the light 34 in a state in which the hood 40 is mounted on the distal end portion 36. The third brightness histogram 106 includes a high-brightness region 106A that is synonymous with the high-brightness region 94A shown in FIG. 12.

The determination unit 54A creates the first brightness histogram 102, the second brightness histogram 104, and the third brightness histogram 106 each time the frame 46 is obtained. The determination unit 54A determines whether or not the first brightness histogram 102 having the same shape, the second brightness histogram 104 having the same shape, and the third brightness histogram 106 having the same shape are continuously created for the predetermined time or longer. Here, in a case in which the first brightness histogram 102 having the same shape, the second brightness histogram 104 having the same shape, and the third brightness histogram 106 having the same shape are continuously created for the predetermined time or longer, the determination unit 54A determines that the hood 40 is mounted on the distal end portion 36.

In a case in which the first brightness histogram 102 having the same shape, the second brightness histogram 104 having the same shape, and/or the third brightness histogram 106 having the same shape are not created, and in a case in which the creation of the first brightness histogram 102 having the same shape, the second brightness histogram 104 having the same shape, and/or the third brightness histogram 106 having the same shape is interrupted in a time shorter than the predetermined time, the determination unit 54A determines that the hood 40 is not mounted on the distal end portion 36. In this way, the same effect as the effect of the above-described embodiment can be obtained.

In the example shown in FIG. 14, the form example has been described in which the first brightness histogram 102, the second brightness histogram 104, and the third brightness histogram 106 are created, and whether or not the hood 40 is mounted on the distal end portion 36 is specified based on the created first brightness histogram 102, the created second brightness histogram 104, and the created third brightness histogram 106, but the present disclosure is not limited thereto. For example, whether or not the hood 40 is mounted on the distal end portion 36 may be specified using a trained model that can distinguish between the frame 46 obtained in a state in which the hood 40 is not mounted on the distal end portion 36 and the frame 46 obtained in a state in which the hood 40 is mounted on the distal end portion 36.

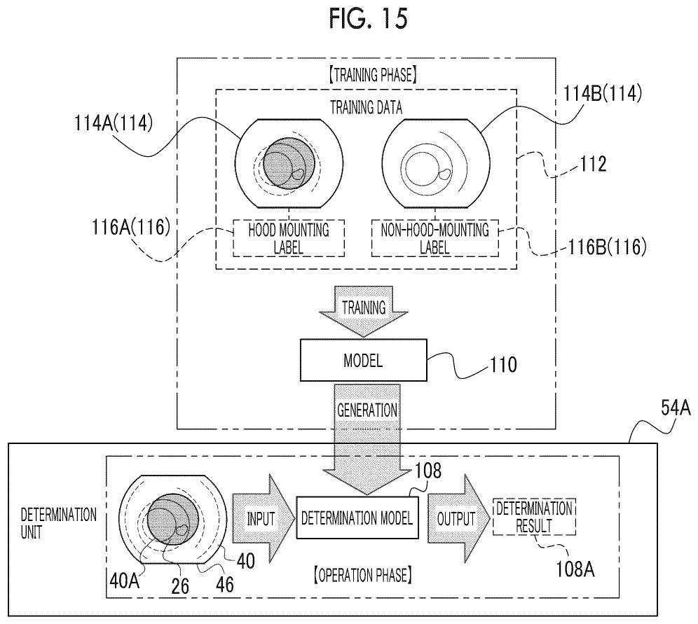

For example, as shown in FIG. 15, a determination model 108 is shown as an example of the trained model. The determination model 108 is obtained by training a model 110 (for example, a neural network) through machine learning using training data 112 in a training phase. The training data 112 includes a plurality of datasets. The dataset is data in which example data 114 and ground truth data 116 are associated with each other.

The example data 114 includes first example data 114A and second example data 114B. The first example data 114A is an image corresponding to the frame 46 obtained by imaging the inside of the large intestine 24 with the camera 30 in a case in which the inside of the large intestine 24 is irradiated with the light 34 in a state in which the hood 40 is mounted on the distal end portion 36. The second example data 114B is an image corresponding to the frame 46 obtained by imaging the inside of the large intestine 24 with the camera 30 in a case in which the inside of the large intestine 24 is irradiated with the light 34 in a state in which the hood 40 is not mounted on the distal end portion 36.

The ground truth data 116 includes a hood mounting label 116A and a non-hood-mounting label 116B. The hood mounting label 116A is a label for specifying that the hood 40 is mounted on the distal end portion 36. The non-hood-mounting label 116B is a label for specifying that the hood 40 is not mounted on the distal end portion 36.

The determination model 108 is generated by training the model 110 through machine learning using the training data 112 composed in this way and optimizing the model 110.

The determination unit 54A inputs the frame 46 to the determination model 108, to cause the determination model 108 to output a determination result 108A. The determination result 108A is information for specifying whether or not the hood 40 is mounted on the distal end portion 36. The determination unit 54A determines whether or not the hood 40 is mounted on the distal end portion 36 with reference to the determination result 108A. In this way, the same effect as the effect of the above-described embodiment can be obtained.

In the example shown in FIG. 15, the form example has been described in which whether or not the hood 40 is mounted on the distal end portion 36 is specified by using the determination model 108, but the present disclosure is not limited thereto. For example, in a case in which specifying information for specifying whether or not the hood 40 is mounted on the distal end portion 36 is given from the outside (for example, the doctor 12), whether or not the hood 40 is mounted on the distal end portion 36 may be specified based on the specifying information given from the outside.

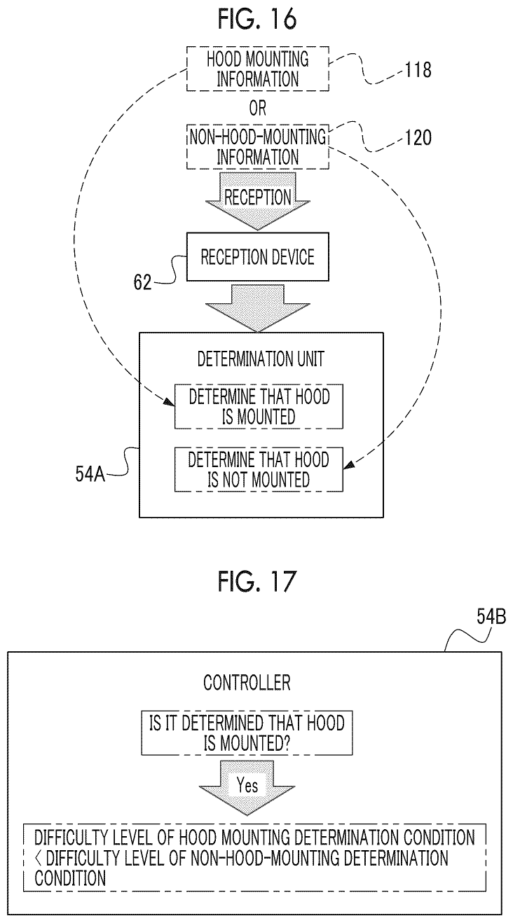

In the example shown in FIG. 16, a form example is shown in which hood mounting information 118 or non-hood-mounting information 120 is received by the reception device 62. The hood mounting information 118 is information for specifying that the hood 40 is mounted on the distal end portion 36. The non-hood-mounting information 120 is information for specifying that the hood 40 is not mounted on the distal end portion 35.

In a case in which the hood mounting information 118 is received by the reception device 62, the determination unit 54A determines that the hood 40 is mounted on the distal end portion 36. In addition, in a case in which the non-hood-mounting information 120 is received by the reception device 62, the determination unit 54A determines that the hood 40 is not mounted on the distal end portion 36. In this way, the same effect as the effect of the above-described embodiment can be obtained.

FIG. 17 shows a modification example of the processing contents of the controller 54B. As shown in FIG. 17, in a case in which the determination unit 54A determines that the hood 40 is mounted on the distal end portion 36, the controller 54B performs control of setting a difficulty level of a non-hood-mounting determination condition to be higher than a difficulty level of a hood mounting determination condition. The non-hood-mounting determination condition refers to a condition used in a case in which the determination unit 54A determines that the hood 40 is not mounted on the distal end portion 36. The hood mounting determination condition refers to a condition used in a case in which the determination unit 54A determines that the hood 40 is mounted on the distal end portion 36. Examples of setting the difficulty level of the non-hood-mounting determination condition to be higher than the difficulty level of the hood mounting determination condition include a form example in which the time condition used by the determination unit 54A is made stricter (that is, the predetermined time used by the determination unit 54A is lengthened or the predetermined number of frames used by the determination unit 54A is increased), the threshold value TH1 shown in FIG. 13 is increased, the threshold value TH2 shown in FIG. 13 is increased, or the predetermined frequency shown in FIGS. 12 and 14 is increased.

In this way, it is possible to suppress the occurrence of a situation in which the determination unit 54A erroneously determines that the hood 40 is not mounted on the distal end portion 36 although the hood 40 is mounted on the distal end portion 36.

In each of the above-described examples, although the form example has been described in which whether or not the hood 40 is mounted on the distal end portion 36 is specified and the switching from the first brightness B1 to the second brightness B2 is performed regardless of the type of the endoscope 14, the present disclosure is not limited thereto. For example, in a case in which the type of the endoscope 14 is not the type in which the hood 40 is mounted on the distal end portion 36, the specification of whether or not the hood 40 is mounted on the distal end portion 36 and/or the switching of the brightness of the frame 46 from the first brightness B1 to the second brightness B2 may not be performed.

In the above-described embodiment, although the form example has been described in which one type of endoscope 14 is used, this is merely an example, and a valid state in which the hood mounting control is valid and an invalid state in which the hood mounting control is invalid may be switched in accordance with the type of the endoscope 14.

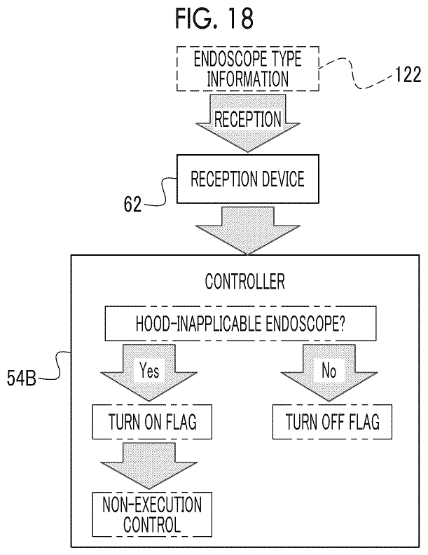

In the example shown in FIG. 18, a form example is shown in which endoscope type information 122 is received by the reception device 62. The endoscope type information 122 refers to information for specifying the type of the endoscope 14. Examples of the type of the endoscope 14 include a variable magnification endoscope having an optical magnification change function (in other words, a magnifying endoscope), a treatment endoscope that can use a treatment tool, a general-purpose endoscope that is used generally, and a bronchoscope. In general, the hood 40 is used for the variable magnification endoscope and the treatment endoscope, but the hood 40 is not used for the general-purpose endoscope and the bronchoscope.

Therefore, in the example shown in FIG. 18, the endoscope type information 122 is received by the reception device 62, and the controller 54B refers to the endoscope type information 122 received by the reception device 62 to determine whether or not the endoscope 14 used in the endoscope system 10 is an endoscope to which the hood 40 cannot be applied (hereinafter, referred to as a “hood-inapplicable endoscope”). In a case in which it is determined that the endoscope 14 used in the endoscope system 10 is the hood-inapplicable endoscope, the controller 54B turns on a flag indicating that the endoscope 14 used in the endoscope system 10 is an endoscope to which the hood 40 is inapplicable (hereinafter, simply referred to as a “flag”). In addition, in a case in which it is determined that the endoscope 14 used in the endoscope system 10 is not the hood-inapplicable endoscope, the controller 54B turns off the flag.

In a case in which the flag is turned on, the controller 54B performs non-execution control, and in a case in which the flag is turned off, the controller 54B does not perform the non-execution control. The non-execution control refers to control of not causing the determination unit 54A to execute the determination of whether or not the hood 40 is mounted on the distal end portion 36 and not causing the controller 54B to execute the switching of the brightness of the frame 46 from the first brightness B1 to the second brightness B2.

In this way, it is possible to prevent the useless processing of specifying whether or not the hood 40 is mounted on the distal end portion 36 or switching the brightness of the frame 46 from the first brightness B1 to the second brightness B2 although the type of the endoscope 14 used in the endoscope system 10 is not the type in which the hood 40 is mounted on the distal end portion 36.

It should be noted that, here, although the form example has been described in which the specification of whether or not the hood 40 is mounted on the distal end portion 36 and the switching of the brightness of the frame 46 from the first brightness B1 to the second brightness B2 is not performed, this is merely an example, and the specification of whether or not the hood 40 is mounted on the distal end portion 36 or the switching of the brightness of the frame 46 from the first brightness B1 to the second brightness B2 may not be performed.

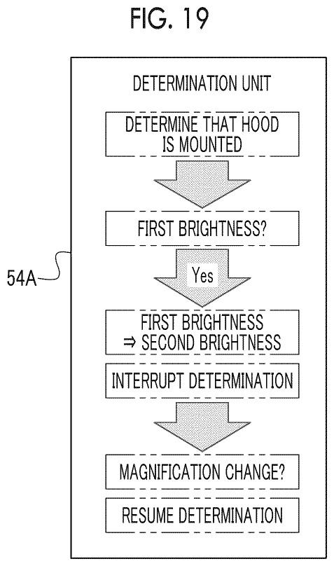

FIG. 19 shows a modification example of the processing contents of the determination unit 54A. As shown in FIG. 19, the determination unit 54A determines whether or not the brightness of the frame 46 is the first brightness B1 on the condition that it is determined that the hood 40 is mounted on the distal end portion 36. Here, in a case in which the determination unit 54A determines that the brightness of the frame 46 is the first brightness B1, the brightness of the frame 46 is switched from the first brightness B1 to the second brightness B2. Then, the determination unit 54A interrupts the determination of whether or not the hood 40 is mounted on the distal end portion 36. In addition, the determination unit 54A resumes the determination of whether or not the hood 40 is mounted on the distal end portion 36 on the condition that the magnification is changed by the variable magnification optical system 66. In other words, the second brightness B2 is maintained from switching of the brightness of the frame 46 from the first brightness B1 to the second brightness B2 to the change in the magnification by the variable magnification optical system 66.

As described above, in the example shown in FIG. 19, in a case in which the brightness of the frame 46 is switched from the first brightness B1 to the second brightness B2 and the determination of whether or not the hood 40 is mounted on the distal end portion 36 is interrupted, the determination of whether or not the hood 40 is mounted on the distal end portion 36 is resumed on the condition that the magnification change is performed by the variable magnification optical system 66. Accordingly, it is possible to prevent the brightness of the frame 46 from becoming inappropriate brightness due to a situation in which the hood 40 is shown in the frame 46 or is not shown in the frame 46 by the magnification change by the variable magnification optical system 66. In addition, in the example shown in FIG. 19, the second brightness B2 is maintained from the switching of the brightness of the frame 46 from the first brightness B1 to the second brightness B2 to the magnification change by the variable magnification optical system 66. Therefore, it is possible to prevent the first brightness B1 and the second brightness B2 from being frequently switched due to an erroneous determination performed by the determination unit 54A as to whether or not the hood 40 is mounted on the distal end portion 36.

It should be noted that, in the example shown in FIG. 19, the form example has been described in which the second brightness B2 is maintained from switching of the brightness of the frame 46 from the first brightness B1 to the second brightness B2 to the magnification change by the variable magnification optical system 66, but this is merely an example, and the second brightness B2 may be maintained from switching of the brightness of the frame 46 from the first brightness B1 to the second brightness B2 to a case in which the specific condition is satisfied. Examples of the specific condition include a condition in which an event in which the need to maintain the second brightness B2 is eliminated has occurred. Examples of the event in which the need to maintain the second brightness B2 is eliminated include an event in which the endoscopy ends, an event in which an instruction to release the second brightness B2 is received by the reception device 62, and the like.

The hood mounting control may be valid in a case in which the magnification of the magnification change by the variable magnification optical system 66 is a first magnification (that is, the same magnification), and may be invalid in a case in which the magnification of the magnification change by the variable magnification optical system 66 is a second magnification greater than the first magnification (that is, in a case of zooming in) or in a case in which the magnification of the magnification change by the variable magnification optical system 66 is a third magnification less than the first magnification (that is, in a case of zooming out). In this way, it is possible to prevent the brightness of the frame 46 from becoming inappropriate brightness due to a situation in which the hood 40 is shown in the frame 46 or is not shown in the frame 46 due to the magnification change by the variable magnification optical system 66.

The content of the hood mounting control may vary in accordance with the type of the instrument mounted on the distal end portion 36. For example, a ratio between the degree in which the average brightness 78 is brought closer to the target average brightness 82 and the degree in which the peak brightness 80 is brought closer to the target peak brightness 84 may be made different between a case in which the instrument mounted on the distal end portion 36 is the hood 40 and a case in which the instrument mounted on the distal end portion 36 is the external treatment tool.

In the above-described embodiment, a magnitude relationship between the target average brightness 74 and the target average brightness 82 is not described, but the target average brightness 82 may be higher than the target average brightness 74. Further, the target peak brightness 84 may be higher than the target peak brightness 76. In addition, a degree in which the target average brightness 82 is set to be higher than the target average brightness 74 may be changed in accordance with the type of the instrument mounted on the distal end portion 36, and a degree in which the target peak brightness 84 is set to be higher than the target peak brightness 76 may be changed in accordance with the type of the instrument mounted on the distal end portion 36.

In the above-described embodiment, the form example has been described in which the brightness control processing is executed by the computer 50, but the present disclosure is not limited thereto, and at least some processing included in the brightness control processing may be executed by a device provided outside the computer 50. In addition, at least some processing included in the brightness control processing may be implemented by network computing such as cloud computing, fog computing, edge computing, or grid computing.

In the above-described embodiment, the form example has been described in which the brightness control program 68 is stored in the storage 58, but the present disclosure is not limited thereto. For example, the brightness control program 68 may be stored in a portable computer-readable non-transitory storage medium, such as an SSD or a USB memory. The brightness control program 68, which is stored in the non-transitory storage medium, is installed in the computer 50 of the endoscope system 10. The processor 54 executes the brightness control processing in accordance with the brightness control program 68.

In addition, the brightness control program 68 may be stored in a storage device of another computer, a server, or the like that is connected to the endoscope system 10 via the network, and the brightness control program 68 may be downloaded and installed in the computer 50 in response to a request from the endoscope system 10.

It is not necessary to store the entire brightness control program 68 in a storage device of another computer or a server device connected to the endoscope system 10 or to store the entire brightness control program 68 in the storage 58, and a part of the brightness control program 68 may be stored.

Various processors shown below can be used as the hardware resource for executing the brightness control processing. Examples of the processor include a CPU which is a general-purpose processor functioning as the hardware resource for executing the brightness control processing by performing software, that is, a program. In addition, an example of the processor is a dedicated electric circuit which is a processor having a dedicated circuit configuration designed to execute specific processing, such as an FPGA, a PLD, or an ASIC. A memory is built in or connected to any processor, and any processor executes the brightness control processing by using the memory.

The hardware resource for executing the brightness control processing may be configured by one of the various processors or by a combination of two or more processors of the same type or different types (for example, a combination of a plurality of FPGAs or a combination of a CPU and an FPGA). Further, the hardware resource for executing the brightness control processing may be one processor.

As a configuring example of one processor, first, there is a form in which one processor is configured by a combination of one or more CPUs and software and the processor functions as the hardware resource for executing the brightness control processing. Secondly, as represented by SoC, there is a form in which a processor that implements the functions of the entire system including a plurality of hardware resources for executing the brightness control processing with one IC chip is used. As described above, the brightness control processing is implemented by using one or more of the various processors as the hardware resources.

Further, as the hardware structure of the various processors, specifically, an electronic circuit in which circuit elements, such as semiconductor elements, are combined can be used. Furthermore, the above-described brightness control processing is merely an example. Therefore, it goes without saying that unnecessary steps may be deleted, new steps may be added, or the processing order may be changed, within a range that does not deviate from the gist of the present disclosure.

The above-described contents and the above-shown contents are the detailed description of the parts according to the present disclosure, and are merely examples of the present disclosure. For example, the descriptions of the configurations, the functions, the operations, and the effects are the descriptions of the examples of the configurations, the functions, the operations, and the effects of the parts according to the present disclosure. Accordingly, it goes without saying that unnecessary parts may be deleted, new elements may be added, or replacements may be made with respect to the above-described contents and the above-shown contents within a range that does not deviate from the gist of the present disclosure. Further, in order to avoid confusion and to facilitate understanding of the parts according to the present disclosure, the description of common technical knowledge or the like, which does not particularly require the description for enabling the implementation of the present disclosure, is omitted in the above-described contents and the above-shown contents.

All of the documents, the patent applications, and the technical standards described in the present specification are incorporated into the present specification by reference to the same extent as in a case in which each of the documents, the patent applications, and the technical standards are specifically and individually stated to be described by reference.

In regard to the above-described embodiment, the supplementary notes will be further disclosed as follows.

### SUPPLEMENTARY NOTE

A control device used for an endoscope having a distal end portion for emitting light, the control device comprising: a processor, in which the processor performs brightness control of bringing brightness of an image obtained by imaging an inside of a body with the endoscope in a state in which the inside of the body is irradiated with the light, closer to target brightness, the brightness control is classified into first brightness control and second brightness control, the first brightness control is performed in a case in which an instrument is not mounted on the distal end portion, the second brightness control is performed in a case in which the instrument is mounted on the distal end portion, and is different from the first brightness control, and the processor detects the instrument mounted on the distal end portion based on a feature value of the image, and switches from the first brightness control to the second brightness control on a condition that the instrument is detected.

Explanation of References

What is claimed is:

1. A control device used for an endoscope having a distal end portion for emitting light, the control device comprising:
a processor,
wherein the processor performs brightness control of bringing brightness of an image obtained by imaging an inside of a body with the endoscope in a state in which the inside of the body is irradiated with the light, closer to target brightness,
the brightness control is classified into first brightness control and second brightness control,
the first brightness control is performed in a case in which an instrument is not mounted on the distal end portion, and
the second brightness control is performed in a case in which the instrument is mounted on the distal end portion, and is different from the first brightness control.

2. The control device according to claim 1,
wherein the first brightness control or the second brightness control is performed based on a result of comparing a light measurement value of a first image obtained by imaging the inside of the body with the endoscope in a state in which the inside of the body is irradiated with the light, with a target light measurement value, and
a degree of increase in brightness of a second image obtained later than the first image by imaging the inside of the body with the endoscope in a state in which the inside of the body is irradiated with the light is greater in the second brightness control than in the first brightness control.

3. The control device according to claim 2,
wherein the brightness of the second image is increased by lengthening an exposure time with the endoscope in a case in which the imaging for obtaining the second image is performed, increasing a light quantity of the light in a case in which the imaging for obtaining the second image is performed, or increasing a degree of an influence of image processing for brightness adjustment on the second image in a case in which the image processing is performed on the second image.

4. The control device according to claim 3,
wherein the image processing is performed on a specific region of the second image, and
the specific region is determined in accordance with the instrument.

5. The control device according to claim 2,
wherein the brightness control is performed based on a plurality of light measurement values obtained by measuring light of the first image using a plurality of light measurement methods, and
a degree of use of the plurality of light measurement values is different between the first brightness control and the second brightness control.

6. The control device according to claim 5,
wherein the plurality of light measurement values include an average light measurement value of the first image, and
a degree of use of the average light measurement value in the second brightness control is greater than a degree of use of the average light measurement value in the first brightness control.

7. The control device according to claim 2,
wherein the brightness control is performed based on the light measurement value of the first image,
the light measurement value is obtained for each divided region in which the first image is divided by a division method different between the first brightness control and the second brightness control, and
magnitude of the light measurement values used in the first brightness control and the second brightness control is magnitude in accordance with the divided region.

8. The control device according to claim 7,
wherein the division method used in the second brightness control is determined in accordance with the instrument.

9. The control device according to claim 2,
wherein the brightness control is performed based on the light measurement value of the first image, and
the light measurement value used in the second brightness control is obtained from an inner region of the first image as compared with the light measurement value used in the first brightness control.

10. The control device according to claim 1,
wherein the endoscope is a variable magnification endoscope, and
the second brightness control is valid in a case in which a magnification of the variable magnification endoscope is a first magnification, and is invalid in a case in which the magnification is a second magnification greater than the first magnification or a third magnification less than the first magnification.

11. The control device according to claim 1,
wherein a valid state in which the second brightness control is valid and an invalid state in which the second brightness control is invalid are switched in accordance with a type of the endoscope.

12. The control device according to claim 1,
wherein the brightness of the image is changed stepwise in a case in which the first brightness control and the second brightness control are switched from one to the other.

13. The control device according to claim 12,
wherein the stepwise change in the brightness of the image is achieved by lengthening, stepwise, an exposure time with the endoscope in a case in which the imaging for obtaining the image is performed, increasing, stepwise, a light quantity of the light in a case in which the imaging for obtaining the image is performed, or increasing, stepwise, a degree of an influence of image processing for brightness adjustment on the image in a case in which the image processing is performed on the image.

14. The control device according to claim 1,
wherein a content of the second brightness control varies in accordance with a type of the instrument.

15. The control device according to claim 1,
wherein the instrument is a tubular hood having an opening.

16. The control device according to claim 1,
wherein the instrument has optical characteristics that cause halation caused by the light.

17. The control device according to claim 1,
wherein the target brightness used in the second brightness control is higher than the target brightness used in the first brightness control.

18. An endoscope system comprising:
the control device according to claim 1; and
the endoscope.

19. An operation method of a control device used for an endoscope having a distal end portion for emitting light, the operation method comprising:
performing brightness control of bringing brightness of an image obtained by imaging an inside of a body with the endoscope in a state in which the inside of the body is irradiated with the light, closer to target brightness,
wherein the brightness control is classified into first brightness control and second brightness control,
the first brightness control is performed in a case in which an instrument is not mounted on the distal end portion, and
the second brightness control is performed in a case in which the instrument is mounted on the distal end portion, and is different from the first brightness control.

20. A non-transitory computer-readable storage medium storing a program executable by a computer used for an endoscope having a distal end portion for emitting light, to execute a process comprising:
performing brightness control of bringing brightness of an image obtained by imaging an inside of a body with the endoscope in a state in which the inside of the body is irradiated with the light, closer to target brightness,
wherein the brightness control is classified into first brightness control and second brightness control,
the first brightness control is performed in a case in which an instrument is not mounted on the distal end portion, and
the second brightness control is performed in a case in which the instrument is mounted on the distal end portion, and is different from the first brightness control.
